(19) United States
(12) Patent Application Publication
(10) Pub. No.: US 2005/0261246 A1
(43) Pub. Date: Nov. 24, 2005

(54) Methods for treating inflammatory and autoimmune diseases

(75) Inventors: Yung Chang, Tempe, AZ (US); Dianne Lorton, Avondale, AZ (US); Cheri Lubahn, Glendale, AZ (US); George R. Pettit, Paradise Valley, AZ (US); David Wilson, Phoenix, AZ (US); Olga Ananieva, Tempe, AZ (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: The Arizona Board of Regents

(21) Appl. No.: 11/071,994

(22) Filed: Mar. 4, 2005

Related U.S. Application Data
(60) Provisional application No. 60/550,759, filed on Mar. 5, 2004.
(60) Provisional application No. 60/553,189, filed on Mar. 15, 2004.
(60) Provisional application No. 60/587,928, filed on Jul. 14, 2004.
(60) Provisional application No. 60/589,109, filed on Jul. 19, 2004.

(51) Int. Cl. A61K 31/675

(52) U.S. Cl. 514/80

(57) ABSTRACT

The present invention provides methods for treating inflammatory conditions, rheumatoid diseases, autoimmune conditions, and conditions associated with bone loss, comprising administering to a subject with an inflammatory condition an amount effective to treat the condition of a compound selected from the group consisting of narcistatin, pancratistatin, pancratastatin-7′ phosphate and pancratastatin-3′,4′ cyclic phosphate, or pharmaceutically acceptable salts thereof.

### CROSS REFERENCE

This application claims the benefit of U.S. Provisional Patent Application Ser. Nos. 60/550,759 filed Mar. 5, 2004; 60/553,189 filed Mar. 15, 2004; 60/587,928 filed Jul. 14, 2004; and 60/589,109 filed Jul. 19, 2004; each of which is incorporated by reference herein in its entirety.

### STATEMENT OF GOVERNMENT INTEREST

The work disclosed herein was supported, at least in part, by grants CA 44344-03-12 and RO1-CA 90441-01-03 from the Division of Cancer Treatment and Diagnosis, National Cancer Institute, DHHS, and by grant ADCRC-9920. Thus, the United States government may have certain rights in the invention

### FIELD OF THE INVENTION

The present invention is related to the fields of therapeutics, inflammation, autoimmunity, arthritis, bone loss, and osteoporosis.

### BACKGROUND OF THE INVENTION

Vascular endothelial growth factor (VEGF) is a potent endothelial cell mitogen in vitro and an angiogenic factor in vivo. In addition to its role in mediating tumor angiogenesis, VEGF also participates in the pathogenesis of many inflammatory diseases, including rheumatoid arthritis (see Giatromanolaki et al., J. Pathol. 194 (2001); Afawape et al., Histol. Histopathol. 17 (2002); and Paleolog et al., Angiogenesis 2 (1998)). It has been reported that the signal transduction pathway that leads to VEGF upregulation overlaps with the pathway involved in inflammation (Paleolog, Arthritis Res. 4 suppl. 3 (2002)). Serum VEGF concentrations are elevated in rheumatoid arthritis and correlate with disease activity. (Sone et al., Life Sci., 69 (2001)).

Nitric oxide (NO) is also a factor that is critical in angiogenesis activity and inflammation. Increased levels of NO correlate with tumor growth and spreading in different experimental cancers. (Lala and Chakraborty, Lancet Oncol. 2:3 (2001)). NO production is a key event in the induction of arthritis in a rat arthritis model, with the level of inducible NO synthase (iNOS) increasing upon pro-inflammatory stimulation by cytokines during inflammation. (Weiberg, Immunol. Res., 22 (2000); Yonekura et al., Nitric Oxide 8 (2003)). NO is elevated in the synovial fluid of rheumatoid arthritis patients. (Borderie et al., J. Rheumatol. 26 (1999)).

Several studies have shown that inhibition of VEGF and iNOS can reduce inflammatory reactions and attenuate disease development (Lu J et al, 2000, J. Immunol; Afuwape et al, 2003, Gene Ther.; Rajas et al, 2003, Eur J Pharmacol; Rojas et al, 2003 Naunyn Schmiedebergs Arch Pharmacol). Thus, a compound that inhibits VEGF and NO could be useful for potential application in treating inflammatory diseases. Therefore, it would be advantageous to identify inhibitors of VEGF and NO for their potential as anti-inflammatory.

### SUMMARY OF THE INVENTION

In one aspect, the present invention provides methods for treating an inflammatory condition comprising administering to a subject with an inflammatory condition an amount effective to treat the inflammatory condition of a compound selected from the group consisting of narcistatin, pancratistatin, pancratastatin-7′ phosphate and pancratastatin-3′,4′ cyclic phosphate, or pharmaceutically acceptable salts thereof.

In another aspect, the present invention provides methods for treating arthritis, comprising administering to a subject with arthritis an amount effective to treat arthritis of a compound selected from the group consisting of narcistatin, pancratistatin, pancratastatin-7′ phosphate and pancratastatin-3′,4′ cyclic phosphate, or pharmaceutically acceptable salts thereof. In various preferred embodiments, the arthritis comprises rheumatoid arthritis or osteoarthritis.

In a further aspect, the present invention provides methods for reducing bone loss in a subject, comprising administering to a subject at risk of bone loss an amount effective to reduce bone loss of a compound selected from the group consisting of narcistatin, pancratistatin, pancratastatin-7′ phosphate and pancratastatin-3′,4′ cyclic phosphate, or pharmaceutically acceptable salts thereof.

In another aspect, the present invention provides methods for treating one or more disorders selected from the group consisting of osteoporosis, osteoarthritis, Paget's disease, humoral hypercalcemia of malignancy, hypercalcemia from tumors metastatic to bone, and periodontal disease, comprising administering to a subject with one or more of the disorders an amount effective to treat the one or more disorders of a compound selected from the group consisting of sodium narcistatin, pancratistatin, pancratastatin-7′ phosphate and pancratastatin-3′,4′ cyclic phosphate, or pharmaceutically acceptable salts thereof.

In another aspect, the present invention provides methods for treating one or more autoimmune disorders selected from the group consisting of rheumatoid arthritis, juvenile chronic arthritis, Crohn's disease, Sjörgen's disease, systemic lupus erythematosus, and psoriasis.

In another aspect, the present invention provides methods for treating one or more rheumatoid diseases comprising administering an amount effective to treat the one or more rheumatoid diseases of a compound selected from the group consisting of narcistatin, pancratistatin, pancratastatin-7′ phosphate and pancratastatin-3′,4′ cyclic phosphate, or pharmaceutically acceptable salts thereof.

In other aspects, the present invention provides methods for reducing cellular production of VEGF or NO in a subject in need thereof comprising administering to the subject an amount effective to reduce cellular production of VEGF or NO of a compound selected from the group consisting of narcistatin, pancratistatin, pancratastatin-7′ phosphate and pancratastatin-3′,4′ cyclic phosphate, or pharmaceutically acceptable salts thereof.

### DESCRIPTION OF THE FIGURES

FIG. 1 provides the chemical structure of compounds of the invention.

FIG. 2A-C is a graph demonstrating the effect of SNS and pancrastatin on production of VEGF. (A) Effect of SNS on VEGF production was analyzed in two cancer cell lines (both supernatants and cell lysates). (B) The supernatant from control and drug treated H460 cells was analyzed for the level of VEGF (pg/ml). C. Summary of inhibitory effect of SNS and pancratistatin on VEGF production from 3-5 individual experiments. Statistical analyses were performed using one-way ANOVA.

FIG. 3 is a graph demonstrating inhibition of nitric oxide NO production in LPS-stimulated RAW 264.7 cells. Cells were pre-treated with different concentrations of SNS and then followed by stimulation with LPS. C is medium control. LPS, positive control without SNS treatment. The statistical analysis was performed using one-way ANOVA.

FIG. 4 provides data on mean body weights 28 days post immunization from arthritic rats receiving no-treatment, or daily treatments of saline, or sodium narcistatin, or non-arthritic rats receiving daily treatments of saline or sodium narcistatin initiated at disease onset and continued until day 28. Significant differences were observed in body weights of untreated- and vehicle-treated arthritic rats compared to vehicle-treated non-arthritic control rats. However, no significant differences were observed between the starting body weights of the vehicle-treated non-arthritic rats and the ending body weights upon completion of the experiment. Chronic sodium narcistatin treatment significantly decreased body weights in the non-arthritic control animals compared to saline-treated non-arthritic controls by day 28. In contrast, the body weights of the arthritic rats treated with sodium narcistatin were not significantly different than untreated or saline-treated arthritic rats. Values represent the mean body weight in grams±SEM with an N of 8 rats per treatment group. Body weights were analyzed using a one-way ANOVA followed by multiple comparison Bonferroni post hoc testing. @ P<0.001, SNS/M. butyricum vs. Saline/M. butyricum. Abbreviations: SNS, sodium narcistatin; SMB, M. butyricum in sterile saline; CFA, complete Freund's adjuvant; Rx, treatment.

FIG. 5 provides data on mean spleen weights 28 days post-immunization from arthritic rats receiving no treatment, or daily treatments of saline, or sodium narcistatin, or non-arthritic rats receiving daily treatments of saline or sodium narcistatin initiated at disease onset and continued until day 28. There was a significant increase in spleen weight with arthritis development in the untreated and saline-treated arthritic animals compared with the antigen-challenged and saline-treated animals. Spleens from arthritic rats treated with sodium narcistatin were not increased in size compared to saline-treated arthritic rats; thus, sodium narcistatin blocked the increase in spleen weight observed in untreated and vehicle-treated arthritic. In contrast, spleen weights of antigen-challenged non-arthritic rats treated with saline did not differ compared to spleen weights of antigen-challenged non-arthritic rats treated with sodium narcistatin. Values represent the mean spleen weights in grams±SEM with an N of 8 rats per treatment group. Spleen weights were analyzed using a one-way ANOVA followed by multiple comparison Bonferroni post hoc testing. @ P<0.001, SNS/M. butyricum vs. Saline/CFA; P<0.001, SNS/CFA vs. Saline/CFA. Abbreviations: SNS, sodium narcistatin; SMB, M. butyricum in sterile saline; CFA, complete Freund's adjuvant; Rx, treatment.

FIG. 6A provides data on dorsoplantar footpad widths 28 days post immunization from arthritic rats receiving no treatment (□), or daily treatments of saline (▴), or sodium narcistatin (♦) or non-arthritic rats receiving daily treatments of saline (▾) or sodium narcistatin (●) initiated at disease onset and continued until day 28. Treatment of CFA-challenged rats with sodium narcistatin significantly decreased the soft tissue swelling of the dorsoplantar footpads between day 23 and day 28 post-immunization compared to vehicle-treated arthritic rats. No differences were seen in dorsoplantar footpad width between vehicle-treated arthritic rats and their non-treated arthritic controls. No inflammation was apparent in any of the limbs of rats treated with the antigen suspended in sterile saline regardless of whether the rats were treated with vehicle or sodium narcistatin. Values represent the mean footpad widths in mm±SEM with an N of 8 rats per treatment group. Footpads were analyzed using a repeated measure two-way ANOVA followed by multiple comparison Bonferroni post hoc testing * P<0.05, # P<0.01, @P<0.001, SNS vs. Saline-CFA. Abbreviations: SNS, sodium narcistatin; SMB, M. butyricum in sterile saline; CFA, complete Freund's adjuvant; Rx, treatment.

FIG. 6B provides representative photomicrographs of the hind limbs 28 days post-immunization from arthritic rats receiving no-treatment, or daily treatments of saline, or sodium narcistatin or non-arthritic rats receiving daily treatments of saline or sodium narcistatin initiated at disease onset. There was redness and soft tissue swelling indicative of severe inflammation in the hind limbs from untreated and vehicle-treated arthritic rats on day 28. Daily treatment of arthritic rats with sodium narcistatin from day 10 through day 28 dramatically decreased the redness and soft tissue swelling compared to arthritic rats receiving no treatment or the vehicle treatment. Hind limbs from rats immunized with the antigen suspended in sterile saline showed no signs of inflammation regardless of whether the rats were treated with saline or sodium narcistatin. Abbreviations: SNS, sodium narcistatin; SMB, M. butyricum in sterile saline; CFA, complete Freund's adjuvant; Rx, treatment.

FIG. 7A provides mean radiographic scores of the hind limbs 28 days post-immunization from arthritic rats receiving no treatment, or daily treatments of saline, or sodium narcistatin, or non-arthritic rats receiving daily treatments of saline, or sodium narcistatin initiated at disease onset and continued until day 28. Untreated and vehicle-treated arthritic rats treated had radiographic scores indicative of severe inflammation and joint destruction. Sodium narcistatin treatment significantly decreased the mean radiographic score of arthritic rats compared to untreated- or saline-treated arthritic animals. Ankle joint radiographs from non-arthritic control animals had no pathology. Values represent the mean radiographic scores±SEM with an n of 8 rats per treatment group. X-rays were evaluated using a scoring method modified from Ackerman and coworkers (1). Radiographic scores were subjected to a non-parametric ANOVA (Kruskal-Wallis analysis) followed by multiple comparison Dunn post-hoc testing; * P<0.05, SNS/CFA vs. Saline/CFA. Abbreviations: SNS, sodium narcistatin; SMB, M. butyricum in sterile saline; CFA, complete Freund's adjuvant; Rx, treatment.

FIG. 7B provides mean representative radiographs of the hind limbs 28 days post immunization from arthritic rats receiving no-treatment, or daily treatments of saline, or sodium narcistatin or non-arthritic rats receiving daily treatments of saline or sodium narcistatin initiated at disease onset. Joint space narrowing and soft tissue swelling is apparent in all the arthritic animals on day 28. There was significant bone loss, soft tissue swelling, periosteal bone formation, joint space narrowing between the metatarsals and a decrease in bone radiolucency in the arthritic animals receiving no treatment or saline injections on day 28. Sodium narcistatin treatment significantly decreased soft tissue swelling as indicated by the decreased width of the hind limb shadows. There were also decreases in bone destruction (mainly osteoporosis and erosions) and cartilage loss in the sodium narcistatin treated animals compared with the saline-treated arthritic rats. An increase in bone luminescence was also apparent in the sodium narcistatin treated animals compared to untreated and vehicle-treated arthritic rats. Radiographs of hind limbs from rats immunized with the antigen in saline showed no signs of soft tissue swelling or bone/cartilage destruction regardless of whether the rats were treated with saline or sodium narcistatin. Abbreviations: AA, adjuvant-induced arthritis (arthritic); SNS, sodium narcistatin; SMB, M. butyricum in sterile saline; CFA, complete Freund's adjuvant; Rx, treatment.

FIG. 8 is a representative analysis of reduction of splenic myeloid cells in SNS-treated rats, revealed by CD11b staining. A. Myeloid populations can be revealed by forward and size scatter (indicated by arrows), as well as staining of a myeloid marker, CD11b, shown in the histogram. B. Summary of myeloid cells (CD11b+ cells) present in various treatment groups. The statistical analysis was performed using t-test (*, N=4). SMB-saline, saline treated, non-arthritic, mycobacterium challenged; SMB-SNS, SNS treated, non-arthritic, mycobacterium challenged; CFA-saline, arthritic Saline treated; CFA-No-RX, arthritic no treatment (stress control); CFA-SNS, SNS treated arthritic. The experimental groups are the same as those in FIGS. 4-7. The data indicates that there is a reduction of CD11b positive myeloid cells in sodium narcistatin treated animals.

FIG. 9A-H the effect of SNS on hind paw swelling in CAIA mice. A and E: Day 0, B and F: Day 7, C: Day 10 in PBS-treated CAIA, G: Day 10 in SNS-treated CAIA after 3 injections, D: Day 12 in PBS-treated mice, and H: Day 12 in SNS-treated CAIA mice after five injections. SNS treatment was started on day 7 after anti-collagen type II mAb injection and i.p. injections of SNS were continued for 5 consecutive days at a dose of 5 mg/kg.

FIG. 10 is a graph of the data (exemplified in FIG. 9) on CAIA progression and effect of SNS on foot pad measurements in CAIA mice. Solid square: PBS-treated CAIA group (n=5), closed triangle: SNS-treated CAIA group (n=5). SNS treatment (i.p.) was started on day 7 after anti-collagen type II mAb injection, and SNS injections were continued for 5 consecutive days at a dose of 5 mg/kg. Values represent mean±S.E.M. P<0.0001. Statistical analysis was performed using t-test.

FIG. 11A-F shows a histological analysis of the effect of SNS on joint destruction in CAIA. Sections of ankle joints were stained with hematoxylin and eosin (original magnification, ×10) on day 21 after mAb injection. SNS treatment (i.p.) was started on day 7 after anti-collagen type II mAb injection, and SNS injections were continued for 5 consecutive days at a dose of 5 mg/kg. Representative joint sections in non-CAIA group (A and D), PBS-treated CAIA (B and E), and SNS-treated CAIA (C and F) are shown at magnifications of ×10 and ×40, respectively. Reduction of polymorphonuclear infiltration and cartilage and bone destruction in the ankle joint area was observed in SNS-treated CAIA mice compared to the PBS-treated CAIA group.

FIG. 12A-B shows the observed reduction of dendritic cells in Balb/c mice treated with SNS. (A) Reduction of CD11c+/CD40+/CD86+ cells isolated from spleens of non-CAIA Balb/c mice treated i.p. with SNS at 5 mg/kg or with PBS for 5 consecutive days. Spleens were isolated on the next day after the last SNS injection and splenocytes were analyzed using FACS. The percentage value represents CD11c+/CD40+/CD86+ cell percentage out of the entire splenocyte population. (B) No changes were observed for expression of cell surface markers B220 and CD90.2 (Thy-1) in the analyzed splenocytes. Values on X and Y axes represent fluorescence intensity for cell surface markers CD11c and CD40, B220, and CD90.2 (Thy-1), respectively.

FIG. 13 provides data demonstrating TNF-α concentration in spleen culture supernatants of PBS or SNS-treated non-CAIA Balb/c mice. Balb/c mice (n=5) were treated i.p. with SNS at 5 mg/Kg or with PBS for 5 consecutive days. Spleen were isolated on the next day after the last SNS injection and the splenocytes were stimulated with anti-CD3 antibody or LPS for 24 hrs, and supernatant were analyzed for TNF-α production.

FIG. 14 demonstrates inhibition of TNF-α and MCP1 production following LPS stimulation (by injection into the air pouch) in Balb/c mice treated with SNS in an in vivo air-pouch assay to measure cytokine production. The reduction of TNF-alpha (P=0.04) and MCP-1 (P=0.03) in SNS-treated mice upon LPS stimulation. PBS_PBS: PBS followed by PBS; PBS_LPS: PBS followed by LPS (1 μg/ml); SNS_PBS: SNS followed by PBS; SNS_LPS: SNS (5 mg/kg) followed by LPS (1 μg/ml). Results are derived from three independent experiments. Statistical analysis was performed using one-way ANOVA.

### DETAILED DESCRIPTION OF THE INVENTION

All references cited are herein incorporated by reference in their entirety.

As used herein, the singular forms “a”, “an” and “the” include plural referents unless the context clearly dictates otherwise. For example, reference to a “polypeptide” means one or more polypeptides.

In each of the various aspects and embodiments of the invention described below, the term “subject” refers to a mammal, preferably a human subject.

In each of the various aspects and embodiments of the invention described below, the phrase “an amount effective” is an amount that is sufficient to provide the intended benefit of treatment. An effective amount of the compounds that can be employed ranges generally between about 0.01 μg/kg body weight and about 20 mg/kg body weight, preferably ranging between about 0.05 μg/kg and about 10 mg/kg body weight. However dosage levels are based on a variety of factors, including the type of injury, the age, weight, sex, medical condition of the individual, the severity of the condition, the route of administration, and the particular compound employed. Thus, the dosage regimen may vary, but can be determined routinely by a physician using standard methods.

In each of the various aspects and embodiments described below, the term “treat” or “treating” means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

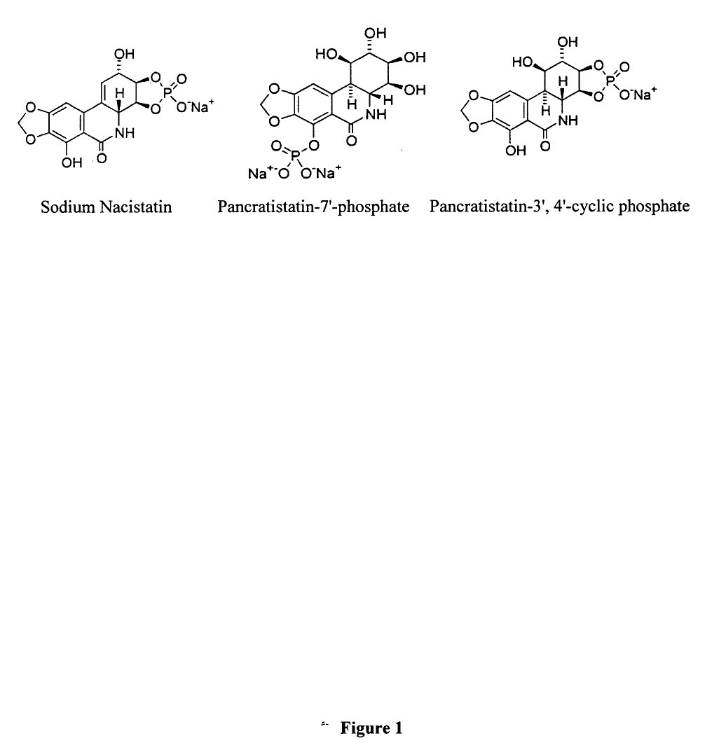

In each of the various aspects and embodiments described below, the terms “narcistatin”, “pancratastatin”, “pancratastatin-7′ phosphate”, and “pancratastatin-3′,4′ cyclic phosphate” include cations thereof, as described, for example, in Pettit et al., J. Nat. Products 66:92-96 (2003), published PCT application WO 2004/052298, and Pettit et al., J. Nat. Products 67:322-327 (2004). Such cations include, but are not limited to, H+, Li+, Na+, K+, Cs+, Mg2+, Ca2+, Zn2+, Mn2+, pyridinium, quinidine, quinine, imidazole, morpohiline, and piperazine. The structures of the named compounds are shown in FIG. 1.

Narciclasine and several related isocarbostyrils isolated (14;15;25;26;28-30) from, for example, the bulbs of Narcissus and Hymenocallis species (Amaryllidaceae) have been found to possess anticancer properties. Narciclasine is not suitable for preclinical testing or clinical applications due to its poor solubility. Sodium narcistatin (SNS) is a synthetic modification of narciclasine that is highly water soluble. Pancratastatin, another compound derived from Hymenocallis littoralis (Pettit, J. Nat. Products, 49 (6), 1986), has been well-characterized, and appears to be more potent than SNS in inhibiting tumor growth. (Pettit et al., J. Nat. Products 56 (10), 1993). Pancratastatin has been found to increase survival rate up to 100% against a flavivirus infection, Japanese encephalitis (Gabrielson et al., J. Natural Products, 55 (11), 1992), and to have activity against the parasite Encephalitozoan intestinalis, a microsporidian causing intestinal and systemic infections in immunocompromised patients (Ouarzane-Amara et al., Antimicrob. Agents Chemother., 45 (12), 2001). Like narciclasine, pancratastatin also has relatively low solubility in biological fluids. As a result, the phosphorylated analog pancratastatin-7′-phosphate was developed. A further derivative of the pancratastatin series is pancratastatin-3′,4′-cyclic phosphate. Each of these compounds share similarity to SNS, including its solubility in biological fluids. Given their comparable inhibition on VEGF production between narcistatin and pancratastin (as demonstrated below), we predict that the derivatives should bear activities similar to those of narcistatin demonstrated herein.

The data disclosed herein provide evidence that narcistatin, and related compounds, are effective anti-inflammatory agents and effective for treating disorders such as rheumatoid arthritis. The data disclosed herein also provide evidence that narcistatin, and related compounds, inhibit cellular production of vascular endothelial growth factor (VEGF), interleukin-1 (IL_{—}1), tumor necrosis factor α (TNFα), and nitric oxide (NO). Without being limited by any specific mechanism, the inventors believe that the various therapeutic effects of narcistatin and related compounds disclosed herein may be due, at least in part, to the inhibition of VEGF, IL-1, TNFα, and/or NO, and that these effects may be working in combination (with respect to at least rheumatoid arthritis) with inhibition of uncontrolled proliferation of the synovium, the connective tissue covering of the joints.

In a first aspect, the present invention provides methods for treating an inflammatory condition comprising administering to a subject with an inflammatory condition an amount effective of a compound selected from the group consisting of narcistatin, pancratistatin, pancratastatin-7′ phosphate and pancratastatin-3′,4′ cyclic phosphate, or pharmaceutically acceptable salts thereof.

The inflammatory process is central to a number of disease states and is the primary defense against infection. Inflammation involves an orchestrated series of events initiated in response to tissue damage. With the initial tissue damage, the innate immune response is activated. This type of immunity is not a pathogen-specific response, but rather functions as the first line of defense against numerous potential threats. Immune cells that are involved in innate immune responses are present, ready to respond prior to the immune challenge and do not required clonal expansion. Phagocytic cells, such as neutrophils and monocytes/macrophages, are key cellular elements in the innate immune responses. After infection or tissue damage, monocytes/macrophages respond rapidly to distinguish self from non-self through expression of cell surface receptors that recognize molecular structures that are shared by large groups of pathogens. These phagocytic cells respond to these types of stimuli by engulfing the bacteria, releasing cytotoxic lysosomal enzymes to kill bacteria and by production proinflammatory cytokines. These cells direct much of the sustained inflammation that occurs in chronic inflammatory diseases. The innate immune response leads to the acquired immune response that involves white blood cell (leukocyte) infiltration into the site of injury, where they are activated and secrete additional mediators of the inflammatory response. If unregulated, the inflammatory state may persist as a condition known as chronic inflammation. In this setting, the mediators produced may amplify the inflammatory response and cause pathology to otherwise normal tissue. Depending upon the body site, such tissue damage may result in chronic diseases such as arthritis (joint inflammation, characterized by pain, stiffness, swelling, and redness), asthma (reversible airway inflammation, often characterized by hyper-responsiveness to various stimuli, coughing, wheezing, shortness of breath, and respiratory distress), emphysema (abnormal permanent enlargement of the airspace; often presents in smokers; characterized by excessive sputum production, cough, wheezing, dyspnea, and fever), ulcerative colitis (chronic inflammatory and ulcerative disease arising in the colonic mucosa; characterized by bloody diarrhea, increased urgency to defecate, and abdominal cramping); and autoimmune diseases including but not limited to rheumatoid arthritis (see below), juvenile chronic arthritis (similar to rheumatoid arthritis, but occurs in children), Crohn's disease (chronic, transmural inflammatory disease that most commonly affects the distal ileum and colon, but may occur in any part of the GI tract-symptoms include chronic diarrhea and abdominal pain, fever, anorexia, weight loss, and a right, lower quadrant mass), Sjörgen's syndrome (characterized by dryness of mouth, eyes, and other mucous membranes, and often associated with rheumatoid disorders sharing certain autoimmune features in which lymphocytes infiltrate mucosal and other tissues), systemic lupus erythematosus (inflammatory connective tissue disorder that can involve joints, kidneys, serous surfaces, and vessel walls; occurs primarily in young women, but also in children: symptoms include arthralgia, arthritis, joint lesions, joint deformity, cutaneous lesions, pleurisy, and pericarditis), and psoriasis (characterized by dry, scaling papules and plaques, often at the scalp, extensor surface of extremities (such as elbows and knees), the sacral area, buttocks, and penis). (Merck Manual, 17^{th} edition, (1999)) Inflammation also results from traumatic injuries, such as joint or muscle strains (“strains”), sprains, cartilage damage, and orthopedic surgery. Other chronic inflammatory conditions include inflammation along nerve roots (such as in sciatica), and atherosclerosis (an inflammation of the blood vessels).

Thus, in a preferred embodiment of this first aspect of the invention, the inflammatory condition is a chronic inflammatory condition. In further preferred embodiments, the inflammatory condition is selected from the group of disorders or conditions consisting of arthritis, inflammatory bowel disease, asthma, emphysema, ulcerative colitis, rheumatoid arthritis, juvenile chronic arthritis, Crohn's disease, Sjörgen's disease, systemic lupus erythematosus, psoriasis, sciatica, atherosclerosis, infection, strain, sprain, cartilage damage, trauma, and recent orthopedic surgery. In a further preferred embodiment, the subject is symptomatic for the condition being treated.

In a second aspect, the present invention provides methods for treating arthritis, comprising administering to a subject with arthritis an amount effective to treat arthritis of a compound selected from the group consisting of narcistatin, pancratistatin, pancratastatin-7′ phosphate and pancratastatin-3′,4′ cyclic phosphate, or pharmaceutically acceptable salts thereof. In a further preferred embodiment of this second aspect of the invention the “arthritis” comprises rheumatoid arthritis (“RA”). In a further preferred embodiment of this second aspect of the invention the “arthritis” comprises osteoarthritis (“OA”). In a further preferred embodiment of the second aspect of the invention, the subject is symptomatic for the condition being treated.

Thus, methods of the invention for treating RA comprise, for example, (a) reducing severity of RA; (b) limiting or preventing development of symptoms characteristic of RA, including but not limited to swelling, pain, inflammation, stiffness, and deformity of affected joints and involved synovial membranes and cartilage; (c) inhibiting worsening of symptoms characteristic of RA, including but not limited to swelling, pain, inflammation, stiffness, and deformity of affected joints and involved synovial membranes and cartilage; (d) limiting or preventing recurrence of RA in patients that have previously had RA; and (e) limiting or preventing recurrence of RA symptoms in patients that were previously symptomatic for RA, including but not limited to symptomatic for swelling, pain, inflammation, stiffness, bone loss and deformity of affected joints and involved synovial membranes and cartilage.

Similarly, methods of the invention for treating OA comprise, for example, (a) reducing severity of OA; (b) limiting or preventing development of symptoms characteristic of OA, including but not limited to pain, inflammation, joint deterioration, loss of bone density, loss of movement, joint stiffness or swelling, joint snapping, bony growths at the joints and/or abnormal angulation, cartilage thinning and/or damage, deformity, and limping; (c) inhibiting worsening of symptoms characteristic of symptoms characteristic of OA, including but not limited to pain, inflammation, joint deterioration, loss of bone density, loss of movement, joint stiffness or swelling, joint snapping, bony growths at the joints and/or abnormal angulation, cartilage thinning and/or damage, deformity, and limping; (d) limiting or preventing recurrence of OA in patients that have previously had OA; and (e) limiting or preventing recurrence of OA symptoms in patients that were previously symptomatic for OA, including but not limited to pain, inflammation, joint deterioration, loss of bone density, loss of movement, joint stiffness or swelling, joint snapping, bony growths at the joints and/or abnormal angulation, cartilage thinning and/or damage, deformity, and limping.

In a third aspect, the present invention provide methods for treating one or more disorders or traumas selected from the group consisting of arthritis, inflammatory bowel disease, asthma, emphysema, ulcerative colitis, rheumatoid arthritis, juvenile chronic arthritis, Crohn's disease, Sjörgen's disease, systemic lupus erythematosus, psoriasis, sciatica, atherosclerosis, infection, strain, sprain, cartilage damage, trauma, and recent orthopedic surgery, comprising administering to a subject with the one or more disorders or traumas an amount effective to treat the one or more disorders or traumas of a compound selected from the group consisting of narcistatin, pancratistatin, pancratastatin-7′ phosphate and pancratastatin-3′,4′ cyclic phosphate, or pharmaceutically acceptable salts thereof. Each of these disorders or traumas is caused, at least in part, by an excessive inflammatory response, as discussed above, and thus treatment using the recited compounds can be used, for example, to lessen the inflammatory response and thus to treat the disorder or trauma. In a further preferred embodiment, the subject is symptomatic for the condition being treated.

Inflammation is the hallmark of many diseases, with the prototypical inflammatory diseases being autoimmune diseases, which include those autoimmune disorders described above. Such chronic diseases are characteristically relapsing and remitting in nature and current treatment is inadequate. (See, for example, U.S. Patent Application Publication No. 20050032686, published Feb. 10, 2005.)

Thus, in a fourth aspect, the present invention provides methods for treating one or more autoimmune disorders selected from the group consisting of rheumatoid arthritis juvenile chronic arthritis, Crohn's disease, Sjörgen's syndrome, systemic lupus erythematosus, and psoriasis, comprising administering to a subject with one or more autoimmune disorders an amount effective to treat the one or more autoimmune disorder of a compound selected from the group consisting of narcistatin, pancratistatin, pancratastatin-7′ phosphate and pancratastatin-3′,4′ cyclic phosphate, or pharmaceutically acceptable salts thereof

The results presented below demonstrate that the compounds used herein can limit bone loss in various animal disease models. There are a variety of disorders that result in loss of bone density. Such “bone loss disorders” include, but are not limited to osteoporosis, Paget's disease, humoral hypercalcemia of malignancy, hypercalcemia from tumors metastatic to bone, and periodontal disease. (See, for example, U.S. Pat. No. 5,830,850).

Thus, in a fifth aspect, the present invention provides methods for reducing bone loss in a subject, comprising administering to a subject at risk of bone loss an amount effective to reduce bone loss of a compound selected from the group consisting of narcistatin, pancratistatin, pancratastatin-7′ phosphate and pancratastatin-3′,4′ cyclic phosphate, or pharmaceutically acceptable salts thereof. Examples of subjects at risk of bone loss are those subjects over the age of fifty who have fractured a bone; those subjects who have lost more than one inch in their height as they have aged; post-menopausal women and women on hormone replacement therapy; as well as those diagnosed with or who previously suffered from a “bone loss disorder.” Thus, in a preferred embodiment of this fifth aspect of the invention, the subject is selected from the group consisting of those over the age of fifty that have suffered a bone fracture; that have lost more than one inch in their height as they aged; post-menopausal women; women on hormone replacement therapy; and those subjects that suffer from one or more conditions selected from the group consisting of osteoporosis, osteoarthritis, Paget's disease, humoral hypercalcemia of malignancy, hypercalcemia from tumors metastatic to bone, and periodontal disease. In a further preferred embodiment, the subject is symptomatic for the condition being treated

In a sixth aspect, the present invention provides methods for treating one or more disorders selected from the group consisting of osteoporosis, osteoarthritis, Paget's disease, humoral hypercalcemia of malignancy, hypercalcemia from tumors metastatic to bone, and periodontal disease, comprising administering to a subject with one or more of the disorders an amount effective to treat the one or more disorders of a compound selected from the group consisting of narcistatin, pancratistatin, pancratastatin-7′ phosphate and pancratastatin-3′,4′ cyclic phosphate, or pharmaceutically acceptable salts thereof. In a further preferred embodiment, the subject is symptomatic for the condition being treated.

In an exemplary preferred embodiment of the sixth aspect of the invention, the subject suffers from osteoporosis, and thus the methods of the invention comprise, for example, (a) reducing severity of osteoporosis; (b) limiting or preventing development of symptoms characteristic of osteoporosis, including but not limited to fracture of vertebrae, wrists, or hips; periodontal disease, Dowager's hump, height loss, back pain, neck pain, bone pain or tenderness, stooped posture; (c) inhibiting worsening of symptoms characteristic of osteoporosis, including but not limited to fracture of vertebrae, wrists, or hips; periodontal disease, Dowager's hump, height loss, back pain, neck pain, bone pain or tenderness, stooped posture; (d) limiting or preventing recurrence of osteoporosis in patients that have previously had osteoporosis; and (e) limiting or preventing recurrence of osteoporosis symptoms in patients that were previously symptomatic for osteoporosis, including but not limited to fracture of vertebrae, wrists, or hips; periodontal disease, Dowager's hump, height loss, back pain, neck pain, bone pain or tenderness, stooped posture.

The data disclosed herein demonstrates that the compounds of the invention are useful for treating a rheumatic disease, rheumatoid arthritis, which has both an inflammatory component and is a connective tissue disorder. Thus, the compounds should also be useful for treating other rheumatic diseases targeting other body organs and tissues. Therefore, in a further aspect, the present invention provides methods for treating one or more rheumatoid diseases comprising administering an amount effective to treat the one or more rheumatoid diseases of a compound selected from the group consisting of narcistatin, pancratistatin, pancratastatin-7′ phosphate and pancratastatin-3′,4′ cyclic phosphate, or pharmaceutically acceptable salts thereof. In a preferred embodiment, the rheumatoid disease is selected from the group consisting of ankylosing spondylitis, diffuse idiopathic skeletal hyperostosis, restrictive lung disease, bacterial infections, arthritis, septic bursitis, myositis, lyme disease, erosive arthritis, viral arthritis, arthralgia, Raynaud's syndrome, polymyositis, mixed connective tissue disease, Takayasu arteritis, polyarteritis nodosa, Churg-Strauss syndrome, Wegener's granulomatosis, Schonlein-Henoch Syndrome, cutaneous leukocytoclastic angiitis, Behcet's syndrome, Buerger's disease, Cogan's disease, Kawasaki disease, Sarcoidosis, Hypergamma globulinemni Purpura of Waldenstrom, polychondritis, sarcoidosis, polymyosistis, dermatomyositis juvenile dermatomysosistis, myosistis associated with collagen vascular disease, inclusion body myositis, myosistis associated with eosinophilia, myosistis ossificans, focal myositis, giant cell myositis, rheumatic fever, gouty arthritis, acute arthritis, fibromyalgia, vasculitis, giant cell arteritis, polymyalgia rheumatica, and localized fibrotic disease. In a further preferred embodiment, the subject is symptomatic for the condition being treated.

The data provided below demonstrate that the compounds used in the methods described reduce cellular production of vascular endothelial growth factor. Thus, in a seventh aspect, the present invention provides methods to reduce cellular production of vascular endothelial growth factor in a subject in need thereof comprising administering to the subject an amount effective to reduce cellular production or vascular endothelial growth factor of a compound selected from the group consisting of narcistatin, pancratistatin, pancratastatin-7′ phosphate and pancratastatin-3′,4′ cyclic phosphate, or pharmaceutically acceptable salts thereof. “Subjects in need” of such treatment include, but are not limited to, those with an inflammatory condition. Exemplary inflammatory conditions are as described above. In a further preferred embodiment, the subject is symptomatic for the condition being treated

The data provided below also demonstrate that the compounds used in the methods described reduce cellular production of nitric oxide. Thus, in an eighth aspect, the present invention provides methods to reduce cellular production of nitric oxide in a subject in need thereof comprising administering to the subject an amount effective to reduce cellular production of nitric oxide of a compound selected from the group consisting of narcistatin, pancratistatin, pancratastatin-7′ phosphate and pancratastatin-3′,4′ cyclic phosphate, or pharmaceutically acceptable salts thereof. “Subjects in need” of such treatment include, but are not limited to, those with an inflammatory condition. Exemplary inflammatory conditions are as described above. In a further preferred embodiment, the subject is symptomatic for the condition being treated.

The data provided below also demonstrate that the compounds used in the methods described reduce cellular production of proinflammatory cytokines, such as IL-1, MCP, and TNF α. Thus, in a ninth aspect, the present invention provides methods to reduce IL-1, MCP and/or TNFα production in a subject in need thereof, comprising administering to the subject an amount effective to reduce IL-1, MCP, and/or TNFα production in the subject of a compound selected from the group consisting of narcistatin, pancratistatin, pancratastatin-7′ phosphate and pancratastatin-3′,4′ cyclic phosphate, or pharmaceutically acceptable salts thereof. “Subjects in need” of such treatment include, but are not limited to, those with an inflammatory condition. Exemplary inflammatory conditions are as described above. In a further preferred embodiment, the subject is symptomatic for the condition being treated.

In a preferred embodiment of each of the aspects and embodiments disclosed above, the compound comprises or consists of narcistatin. It is further preferred that the narcistatin is sodium narcistatin for each of the aspects and embodiments disclosed herein.

The term “pharmaceutically acceptable salts” as used herein in each of the aspects and embodiments of the invention refers to those salts that are within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M. et al., “Pharmaceutical Salts,” J. Pharm. Sci., 1977; 66:1-19 which is incorporated herein by reference.)

The instant compounds can be administered individually or in combination, usually in the form of a pharmaceutical composition. Such compositions are prepared in a manner well known in the pharmaceutical art and comprise at least one active compound.

The compounds of the invention can be administered as the sole active pharmaceutical agent, or they can be used in combination with one or more other agents to treat the particular condition. When administered as a combination, the therapeutic agents can be formulated as separate compositions that are given at the same time or different times, or the therapeutic agents can be given as a single composition.

For example, when treating rheumatoid arthritis, the compounds of the invention can be used in combination with existing treatments, including but not limited to diclofenac, fenuprofen, flubiprofen, ibufprofen, indomethacin, ketoprofen, meclofenamate, nabumetone, naproxen, oxaprozin, piroxicam, sulindac, tolmetin, Cox-2 inhibitors (including but not limited to CELEBREX™, VIOXX™, and BEXTRA™, gold compounds, hydroxychloroquine, sulfasalazine, penacillamine, corticosteroids, pain medications, and cytotoxic or immunsuppressive drugs (including, but not limited to, methotrexate, azathiprine, and cyclosporine).

The pharmaceutical compositions of this aspect of the invention include admixtures of the compounds of the invention, or pharmaceutically acceptable salt thereof, and the one or more other compounds, as well as separate unit dosages of each that are manufactured for combinatorial use. Such separate unit dosages may be administered concurrently or sequentially as determined by the clinician.

The compounds may be made up in a solid form (including granules, powders or suppositories) or in a liquid form (e.g., solutions, suspensions, or emulsions). The compounds of the invention may be applied in a variety of solutions and may be subjected to conventional pharmaceutical operations such as sterilization and/or may contain conventional adjuvants, such as preservatives, stabilizers, wetting agents, emulsifiers, buffers etc.

For administration, the compounds are ordinarily combined with one or more formulation components appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compounds of this invention may be dissolved in saline, water, polyethylene glycol, propylene glycol, carboxymethyl cellulose colloidal solutions, ethanol, tragacanth gum, and/or various buffers. Other formulation components and modes of administration are well known in the pharmaceutical art. The carrier or diluent may include time delay material, such as glyceryl monostearate or glyceryl distearate alone or with a wax, or other materials well known in the art.

The compounds of the invention may be administered by any suitable route, including orally, parentally, by inhalation or rectally in dosage unit formulations containing conventional pharmaceutically acceptable carriers, adjuvants, and vehicles, including liposomes. The term parenteral as used herein includes, subcutaneous, intravenous, intraarterial, intramuscular, intrasternal, intratendinous, intraspinal, intracranial, intrathoracic, infusion techniques, intracavity, or intraperitoneally. In a preferred embodiment, the compounds of the invention are administered orally or parentally.

The compounds may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data.

In a preferred embodiment of each of the above aspects of the invention, the pharmaceutical compositions of the invention are prepared for oral administration. As such, the pharmaceutical composition can be in the form of, for example, a tablet, a hard or soft capsule, a lozenge, a cachet, a dispensable powder, granules, a suspension, an elixir, a liquid, or any other form reasonably adapted for oral administration. The pharmaceutical compositions can further comprise, for example, buffering agents. Tablets, pills and the like additionally can be prepared with enteric coatings. Unit dosage tablets or capsules are preferred. Oral compositions will generally include an inert diluent or an edible carrier and may be compressed into tablets or enclosed in gelatin capsules. Pharmaceutically compatible binding agents and other materials known in the art can be included as part of the composition.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known for example, as described in U.S. Pat. No. 4,522,811.

The active compounds may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

The instant invention may be embodied in other forms or carried out in other ways without departing from the spirit or essential characteristics thereof. The present disclosure and enumerated examples are therefore to be considered as in all respects illustrative and not restrictive, the scope of the invention being indicated by the appended claims, and all equivalencies are intended to be embraced therein. One of ordinary skill in the art would be able to recognize equivalent embodiments of the instant invention, and be able to practice such embodiments using the teaching of the instant disclosure and only routine experimentation.

### EXAMPLES

### Example 1

### SNS Effect on VEGF Production and Lipopolysaccharide (LPS)-Induced NO Secretion

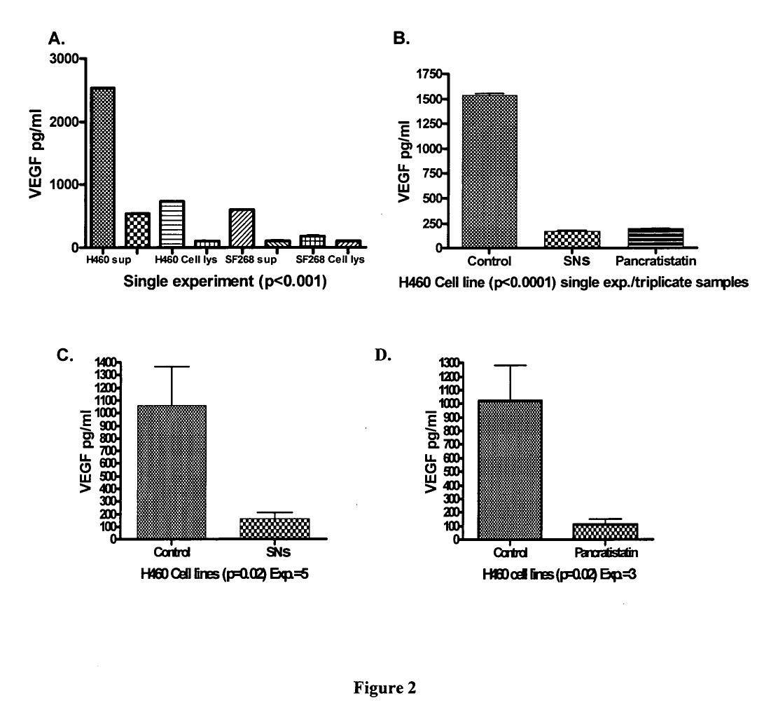

As discussed above, SNS and pancratastatin were originally synthesized as an anti-cancer agent. The inventors also examined the effect of SNS and pancratastatin on the production of VEGF in human cancer cell lines (FIG. 2). VEGF was measured in an ELISA This is one representative experiment to show the inhibitory effect of SNS and pancrastatin on VEGF production in human cancer cell lines. This analysis had been performed at least 3-5 times depending on the cell lines and drug used, with statistical analysis performed using one-way ANOVA. Results showed that SNS significantly suppresses the secretion of VEGF from these cancer cells.

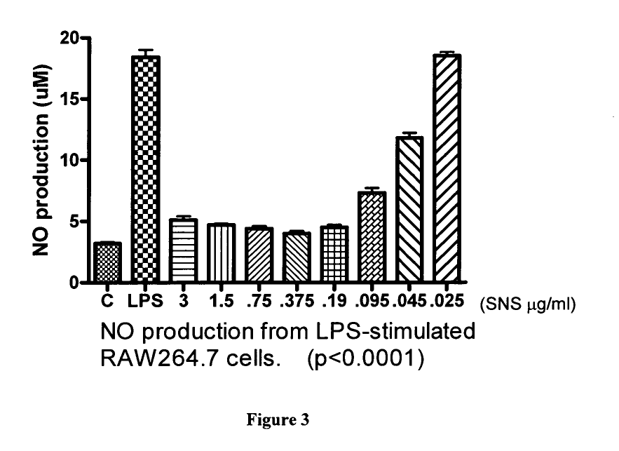

In further tests, we investigated the effect of SNS on the production of NO in a mouse macrophage cell line, RAW264.7. Cells were pre-treated with different concentrations of SNS and subsequently stimulated with 1 ng/ml of LPS. The level of NO production was measured by a spectrophotographic method using the Griess reaction. A concentration-dependent inhibition of NO production was observed, as in FIG. 3. “C” is medium control, and “LPS” is positive control without SNS treatment. This analysis had been performed at least 3-5 times depending on the cell lines and drug used, with statistical analysis performed using one-way ANOVA. Thus, it was found that SNS exerts a direct inhibitory effect on the production of VEGF in tumor cells and NO in an LPS-induced macrophage cell line.

As discussed above, several studies have already shown that inhibition of VEGF and iNOS could reduce inflammatory reactions and attenuate the disease development (Lu J et al, 2000, J. Immunol; Afuwape et al, 2003, Gene Ther.; Rajas et al, 2003, Eur J Pharmacol; Rojas et al, 2003 Naunyn Schmiedebergs Arch Pharmacol). Thus, the inhibitory activity of SNS on both VEGF and NO production in vitro suggested that SNS may act as an inflammatory inhibitor in vivo. The potential of SNS as an anti-inflammatory therapeutic agent was subsequently demonstrated in the arthritic rat model.

### Example 2

### SNS Effect in an Arthritic Rat Model

Summary: Adjuvant-Induced Arthritis (“AA”) was induced by intradermal injection of complete Freund's adjuvant (CFA) into the base of the tail of Lewis rats. Controls received intradermal injections of the arthritogenic antigen, M. butyricum suspended in saline. These rats are antigen-challenged but do not develop AA. AA and control rats were given once-daily intraperitoneal (i.p.) injections of narcistatin (5 mg/Kg/day, in 250 μl sterile saline), vehicle, or no treatment initiated at disease onset and continued through severe disease.

Results. Sodium narcistatin dramatically reduced hind limb inflammation (˜70%), as measured by dorsoplantar width, and bone loss (˜50%), as measured by radiographic analysis, in rats with AA compared to vehicle treated AA rats.

Introduction

Rheumatoid arthritis (RA) is a systemic disease characterized by a chronic inflammation, the loss of bone density, the invasion of the articular cartilage by the synovial membrane, and the deformation of the bones in affected joints. One of the pathological hallmarks of RA is the tumor-like expansion of inflamed synovial tissue, or pannus, into the adjacent articular cartilage and bone which causes much of the damage in the diseased joint (3;9). Histologically, the hyperplastic synovium is infiltrated with neutrophils, monocytes, and lymphocytes, immune cells that direct the ongoing local inflammatory response (reviewed in (4;13)). Synovial invasion and destruction of joint cartilage and bone result from enzymatic degradation of a variety of structural proteins that give the joint its characteristic biomechanical properties. While normal synovial fibroblasts and chondrocytes produce both matrix-degrading proteases (metalloproteases and cysteine proteases) and their inhibitors, in RA the physiological balance is disrupted, resulting in an over production of proteases (4;13). This imbalance can be induced experimentally by proinflammatory cytokines, such as TNF-α and IL-1 (4), suggesting that monocytes and macrophages can regulate this process.

That proinflammatory cytokines, products of monocytic cells, can induce an RA-like disease (16;35) in experimental animals supports a key role for myelomonocytic cells in the production and perpetuation of synovial inflammation in RA. Macrophage infiltration in the synovium correlates with the development of joint erosions (22;23). This is consistent with studies demonstrating systemic activation of macrophages (blood, spleen, and peritoneal cavity) precedes and correlates with arthritis induction and progression (4;22). Furthermore, treatments that target activated macrophages or their products have been the most effective therapeutics in ameliorating the disease (2;4;5;8;10-12;17;18).

TNF-α is recognized as a pivotal cytokine that regulates inflammation and has a major role in disease pathology in RA (10;11). Inhibition of macrophage TNF-α production could explain the decrease in disease severity observed following treatment with narciclasine. Dramatic effects in reducing inflammation and joint destruction after treatment with anti-TNF-α therapies have been observed in murine collagen-induced arthritis (35), transgenic mice that over-express TNF-α (16), and RA patients (10;11).

The present study has examined whether sodium narcistatin can attenuate development of severe AA disease pathology when administered from disease onset through severe disease stages. We report that sodium narcistatin reduced joint inflammation and dramatically decreased bone and cartilage damage in a rat AA model. These findings suggest that sodium narcistatin or other narcistatin cation derivatives (27) can be used as an effective drug therapy for RA patients.

Materials and Methods

Animals

Adult male Lewis rats (200-250 g) were purchased from Charles River Laboratories, Raleigh, N.C. and housed two per cage. The animals were allowed to acclimate to the Sun Health Research Institute's vivarium for 7 days prior to the start of the experiment. The animals were maintained on a 12-h off/on light schedule. For AA rats rodent Diet (Purina Lab Diet 5001) was placed in the bottom of the cage and water was supplied using long-stemmed sipper tubes for easy access to food and water. All rats were observed to eat and drink. Animals were weighed and observed daily to ensure adequate weight gain and good general health. Body weights and footpad measures were started 1 week prior to the first adjuvant injection to acclimate the animals to these mild stresses and obtain baseline data. Other than the development of arthritis, the good health of the animals was maintained throughout the course of the experiments. Protocols for the use and care of the animals in the study were approved prior to beginning the experiments by the Sun Health Research Institute Animal Use and Care Committee and complied with NIH guidelines for the humane use and care of research animals. Dorsoplantar footpad measurements were completed every other day. Prior to sacrifice the animals were given a 1.0 ml intraperitoneal (i.p.) injection of 8% chloral hydrate in sterile saline and radiographs were taken of their hind limbs to assess disease severity. The animals were then sacrificed using an overdose of chloral hydrate.

Chemicals and Adjuvant

Sodium narcistatin was synthesized as previously described (27). CFA was prepared by emulsifying Mycobacterium butyricum (0.03 g dried and heat killed; Difco, Detroit, Mich. in 10 ml sterile mineral oil). M. butyricum (0.03 g) also was suspended in 10 ml sterile saline. The CFA and M. butyricum in saline were prepared by grinding the M. butyricum with a mortar and pestle until the lyophilized bacteria had turned from a light beige to an eggshell white powder. The mineral oil or saline was then slowly worked into the heat-killed bacteria using the mortar and pestle. The suspensions were treated with a sonic dismembraner for 5 min to ensure that the bacterial cell wall remained suspended in the mineral oil or saline until the animal injections. While there is variability in severity of disease development between the batches of adjuvant, there is very little variability within each batch. All animals in each experiment were challenged with the same preparation of adjuvant and 100% of the animals developed arthritis with similar timing of disease onset.

Lewis rats were randomly assigned into five experimental groups of four animals per group. The experimental groups were 1) saline/M. Butyricum suspended in saline (non-arthritic; control for drug and antigen challenge), 2) sodium narcistatin/M. Butyricum suspended in saline (non-arthritic; drug control for antigen challenge), 3) no treatment-CFA (arthritic; control for stress of injections), 4) saline-CFA (arthritic; control for drug treatment), and 5) sodium-narcistatin-CFA (arthritic) treatments. The experiment was completed with an N=4 then repeated with an additional N=4. The data from each group in the first and repeated experiment were compared. No statistical differences between the findings from these repeat experiments were found; therefore, the data was combined to give an N=8 for each experimental group. CFA or saline/M. Butyricum injections were given on experimental day 1. Sodium narcistatin (5 mg/Kg/day, 250 μl) or vehicle (250 μl sterile 0.9% saline) treatments were started on day 10 and continued through day 28 post-immunization. Untreated animals were handled but received no injection. The time point to initiate drug treatments was chosen based on physical symptoms (soft tissue swelling and redness in the hind limbs) representing a time point where disease onset was confirmed.

Assessment of Arthritis

The inflammatory response in the arthritic rats was assessed by routine methods previously described (1). Dorsoplantar width of the hind feet were measured using a Mitutoyo Corporation dial thickness gauge, beginning one week before the day of CFA or M. butyricum in saline administration and continued approximately every other day until sacrifice. The right and left footpads from each animal were averaged together. The individual means for each animal were then averaged within each group and subjected to a repeated measure two-way analysis of variance (ANOVA; P<0.05) with Bonferroni post hoc testing. Radiographs were taken the day of sacrifice using the following settings: 400 nN, 50 kvp, and 0.4 second exposure, at 40 cm and X-OMAT processor. X-rays were evaluated using a grading scale modified from Ackerman and coworkers (1). In short, the radiographs were coded to obscure the treatment groups, and then two independent observers subjectively rated each of the radiographs on the scale: 0 (normal), 1 (slight), 2 (mild), 3 (moderate), and 4 (severe) abnormalities in the tissue without knowledge of the treatment. The radiographs were scored for each of the following characteristics: (1) swelling as indicated by the width of soft tissue shadows and alterations in the normal configuration of the soft tissue planes; (2) osteoporosis as measured by bone density (recognized by increases in radiolucency relative to uninvolved adjacent bone); (3) cartilage loss shown by narrowing of the joint spaces; (4) destruction of bone (erosions) and (5) heterotopic ossification defined as proliferation of new bone tissue (fine ossified lines paralleling normal bone but not contiguous with calcified area of the bone itself). The radiographic scores for each category were added for both hind limbs giving a maximum score of 40, and the individual scores for each animal were then averaged within the treatment groups, expressed as a mean±standard error of the mean (SEM), and subjected to Kruskal-Wallis statistical analysis (non-parametric statistic equivalent to an one-way ANOVA; P<0.05) followed by Dunn post-hoc testing.

Results:

Animal Body and Spleen Weights

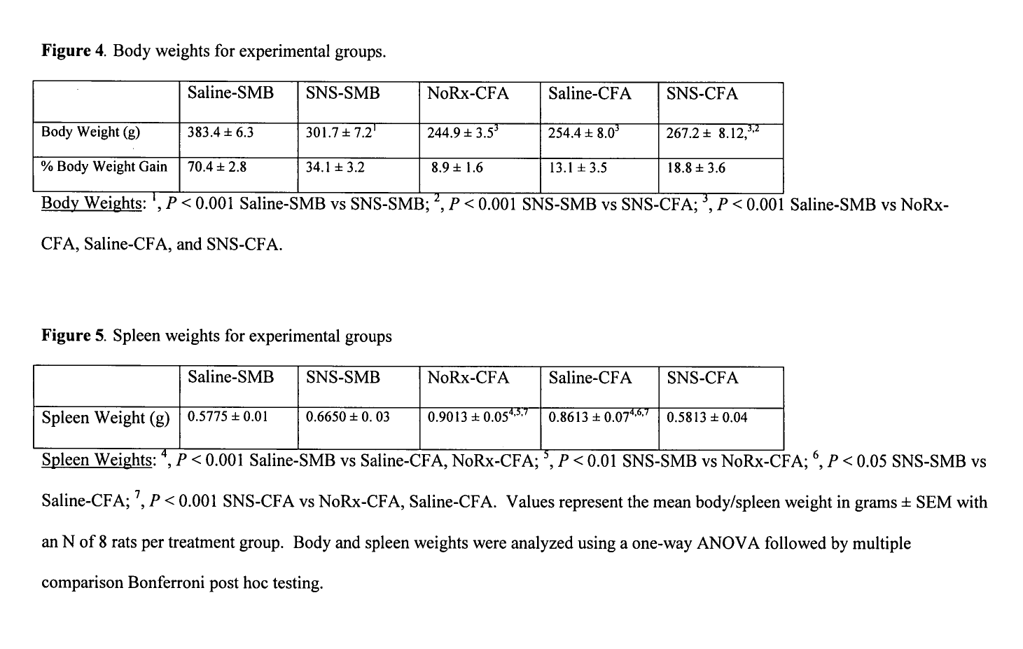

Previous studies from our laboratory have demonstrated that while the arthritic animals are able to maintain their weight with arthritis development, they do not continue to gain weight compared with non-arthritic control animals (20). This is demonstrated by the % body weight gain indicated in FIG. 4. Similarly in this study, body weights from untreated or vehicle arthritic rats did not increase during the course of the experiment; however, they were able to maintain their body weights from the time of disease onset through severe disease development (data not shown). The body weights of the arthritic rats treated with sodium narcistatin were not significantly different than untreated or saline-treated arthritic rats (FIG. 4).

On day 28, spleen weights differed significantly between experimental groups (FIG. 5). There was a significant increase in spleen weight with arthritis development in the untreated and saline-treated arthritic animals compared with the antigen-challenged and saline-treated animals (t=5.180, saline/M. butyricum vs. no treatment-CFA; t=4.540, saline/M. butyricum vs. saline-CFA). Treatment of arthritic rats with sodium narcistatin blocked this increase in spleen weight with arthritis development (t=4.480, Saline-CFA vs. sodium narcistatin-CFA) (FIG. 5). In contrast, spleen weights of antigen-challenged non-arthritic rats treated with saline did not differ compared to antigen-challenged non-arthritic rats treated with narcistatin.

Footpad Measurements

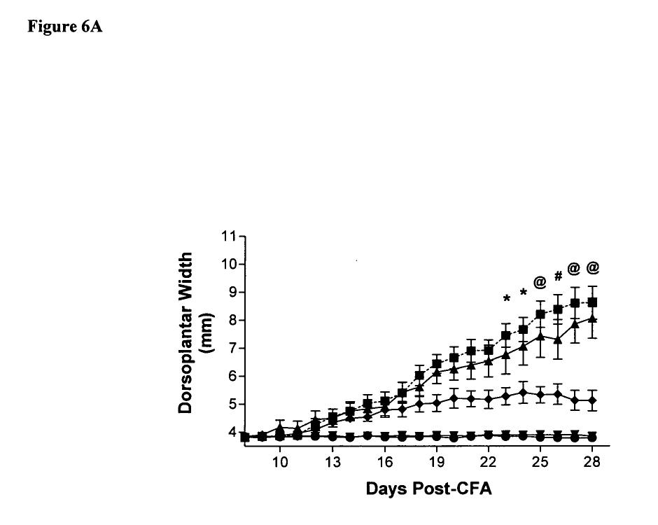

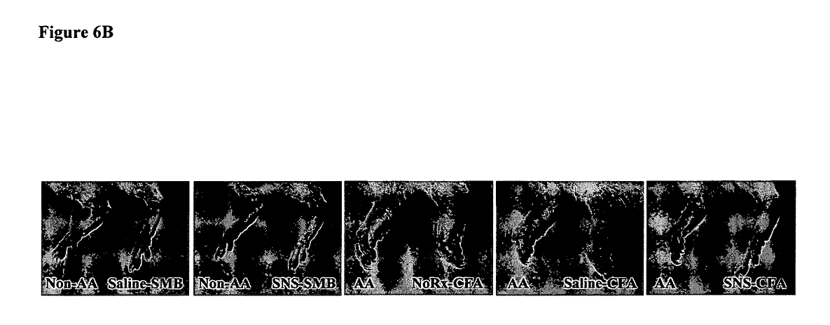

To evaluate the effects of chronic treatment with sodium narcistatin on disease severity, dorsoplantar widths of the hind limbs were measured daily after CFA treatment throughout disease development. Approximately 9-10 days, following base of the tail injection with CFA, the soft tissue swelling became apparent in all CFA-treated rats. The dorsoplantar widths for CFA-challenged arthritic animals continued to increase through the effector phase of the disease (FIG. 6A). Treatment with sodium narcistatin did not change the time of disease onset. Treatment of CFA-challenged rats with sodium narcistatin significantly decreased the soft tissue swelling of the dorsoplantar footpads between day 23 and day 28 post-immunization compared to vehicle-treated arthritic rats (Day 23: t=3.252, P<0.05; Day 24: t=3.556, P<0.05; Day 25: t=4.532, P<0.001; Day 26: t=4.218, P<0.01; Day 27: t=5.898, P<0.001; and Day 28: t=6.350, P<0.001). No differences were seen in dorsoplantar footpad width between vehicle-treated arthritic rats and their non-treated arthritic controls. No inflammation was apparent in any of the hind limbs of rats treated with the antigen suspended in sterile saline regardless of whether the rats were treated with vehicle or sodium narcistatin. There were also no differences in footpad width between the non-arthritic animals treated with vehicle or sodium narcistatin. FIG. 6B shows representative micrographs demonstrating qualitative differences in between hind feet of non-arthritic and arthritic rats treated with vehicle or sodium narcistatin.

Radiographic Scores

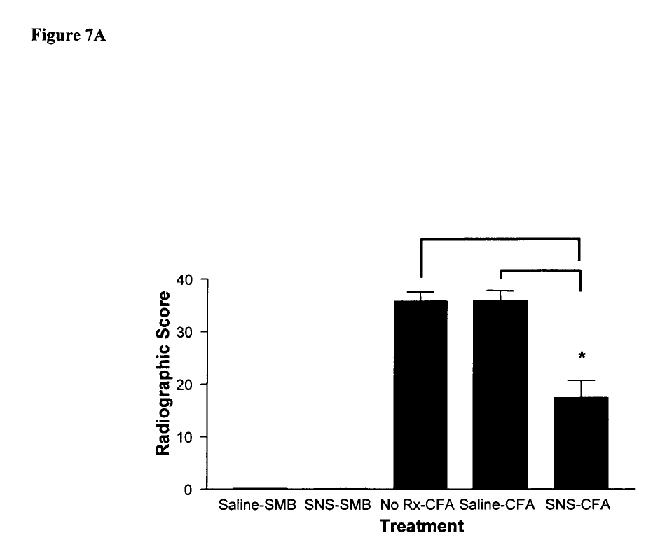

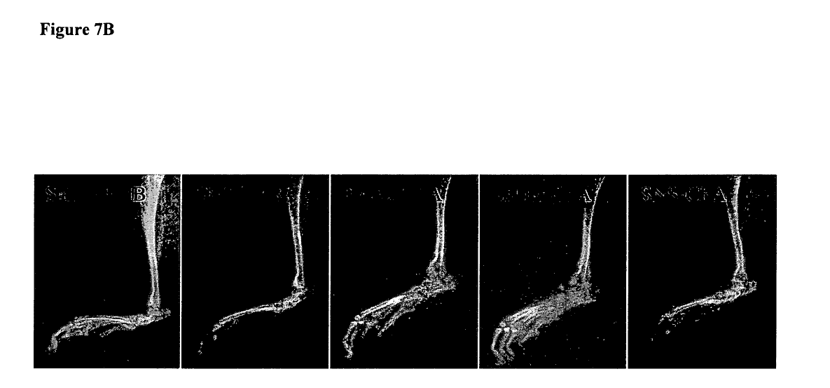

Radiographic analysis of the ankle joints on day 28 revealed destructive joint changes in the adjuvant-challenged groups (FIG. 7A). There was significant bone loss, soft tissue swelling and periosteal bone formation coupled to a narrowing of the joint spaces between the metatarsals and a decrease in bone radiolucency in the arthritic animals receiving no treatment or saline injections on day 28. Arthritic rats treated with sodium narcistatin had significantly lower radiographic scores compared with the arthritic rats treated with vehicle or receiving no treatment (Kruskal-Wallis statistic 34.25; Dunn multiple comparison test 11.69: P<0.05). These effects are illustrated in the representative radiographs pictured in FIG. 7B. Examination of radiographs of arthritic rats treated with sodium narcistatin revealed reduced bone loss, soft tissue swelling, periosteal bone formation, narrowing of the joint spaces and bone density compared to untreated or vehicle-treated arthritic rats. Radiographs of hind limbs from rats immunized with the antigen suspended in saline showed no signs of soft tissue swelling or bone/cartilage destruction regardless of whether the rats were treated with saline or sodium narcistatin (FIG. 7B).

Discussion

The ability of sodium narcistatin, a narciclasine derivative with a water-soluble cyclic phosphate structural modification (27), to attenuate development of severe AA disease pathology was examined in this study. Treatment with sodium narcistatin administered daily from disease onset through severe disease stages reduced joint inflammation and dramatically decreased bone and cartilage damage in a rat AA model. The ability of sodium narcistatin to inhibit soft tissue swelling and joint inflammation is similar to a previous report in which Mikami and coworkers (21) used a different model of AA to demonstrate that prophylactic treatment with narciclasine reduced inflammation. However, Mikami et al. (21) demonstrated that treatment with narciclasine, if given from the time of CFA challenge, was able to inhibit inflammation during the acute inflammatory phase (day 14 post-CFA challenge). The effectiveness of the treatment was lost by the development of severe chronic inflammation (day 21 post-CFA challenge), when treated and untreated arthritic rats were found to have no differences in their footpad volumes. Thus, the ability sodium narcistatin to dramatically inhibit inflammation during chronic disease phases is a new finding. The finding in this study demonstrating that sodium narcistatin can also reduce joint destruction in an animal model that develops an aggressive and severe form of arthritis is novel. Collectively these findings indicate that sodium narcistatin and other narcistatin cation derivatives (27) have both anti-inflammatory and bone sparing properties, which can be developed into effective therapeutic drugs used to treat RA patients.

Sodium narcistatin was very effective in reducing the severe inflammation and joint destruction that develops in the AA model used in this study. Arthritic rats treated with sodium narcistatin tolerated the drug treatment well and no general signs of overt toxicity were apparent based on maintenance of body weights and gross evaluation of internal organs (data not shown) at the experimental end point. However, non-arthritic rats treated with sodium narcistatin that were antigen-challenged with M. butyricum suspended in saline did have reduced body weights after 19 days of drug treatment compared to vehicle treated and immune challenged non-arthritic control rats. This is similar to results seen with narciclasine that demonstrate it inhibits the growth rate of healthy mice (33). Future studies will be undertaken directed at the preclinical development of narcistatin to evaluate the tolerability and risks of sodium narcistatin for potential use in treating RA.

In previous studies from our laboratory, we have demonstrated an increase in whole spleen weight with arthritis development compared to vehicle (mineral oil)-treated rats that do not develop arthritis (20). Interestingly, rats that develop arthritis had greater spleen weights than rats that were immunized with a same antigen that did not develop arthritis suggesting the increase in spleen weight is disease specific. This idea is supported by the extensive reports that spleen derived-immune cells play a significant role in the disease pathology (6;7;19;32;34). Treatment of arthritic rats with sodium narcistatin blocked this disease-specific increase in spleen size. This result is consistent with the known anti-proliferative properties of sodium narcistatin (27). These data suggest that sodium narcistatin could be mediating some of its effects through secondary lymphoid organs. Interestingly, sodium narcistatin did not alter spleen weights from immunized rats that did not develop arthritis, suggesting that sodium narcistatin did not inhibit the immune cell proliferation associated with the antigen challenge. Further studies will be needed to determine the effects of sodium narcistatin on immune cell proliferation, homing, and activation in secondary lymphoid organs.

Future experiments are also required to determine the mechanism(s) by which sodium narcistatin reduce(s) inflammation and joint destruction in AA. Whether these effects of sodium narcistatin are due to its antiproliferative properties is not clear (27). Sodium narcistatin could also be inhibiting expansion of T lymphocytes, monocytes and other immune cells or their products following disease development. In addition to antimitotic properties, Yui and co-workers (36) have reported that narciclasine inhibits lipopolysaccharide (LPS)- or bacteria-induced production of TNF-α by macrophages. TNF-α is recognized as a pivotal cytokine that regulates inflammation and has a major role in disease pathology in RA (11). Inhibition of macrophage TNF-α production could explain the decrease in disease severity observed using narciclasine, as dramatic effects in reducing inflammation and joint destruction following treatment with anti-TNF-α therapies have been observed in murine collagen-induced arthritis (35), transgenic mice that over-express TNF-α (16), and RA patients (8;11;16).

In conclusion, sodium narcistatin treatment from disease onset through development of severe disease dramatically reduced inflammation and joint destruction in AA. Sodium narcistatin treatment was well tolerated at the dose and time course of treatment in arthritic rats and prevented the disease associated increases in spleen weight. The bone sparing effects following sodium narcistatin treatment of arthritic rats is a novel finding. The potent anti-inflammatory effect of treatment with sodium narcistatin after disease onset and through the chronic inflammatory stages is also a new finding. Given that current drug therapies are not effective in preventing bone destruction, these data support further investigation of sodium narcistatin as an anti-rheumatic drug.

Abbreviations for Example 2:

AA=Adjuvant-Induced Arthritis; ANOVA=Analysis of Variance; CIA=Collagen II-Induced Arthritis; CFA=Complete Freund's Adjuvant; i.p.=intraperitoneal; PBS=Phosphate Buffered Saline; RA=Rheumatoid Arthritis; SNS=Sodium narcistatin.

References for Example 2:

(1) Ackerman N R, Rooks W H II, Shott L, Genant H, Maloney P, West E. Effects of naproxen on connective tissue changes in the adjuvant arthritic rat. Arthritis Rheum 1979; 22:1365-74.
(2) Allen J B, Wong H L, Costa G L, Bienkowski M J, Wahl S M. Suppression of monocyte function and differential regulation of IL-1 and IL-1ra by IL-4 contribute to resolution of experimental arthritis. J Immunol 1993; 151:4344-51.
(3) Bresnihan B, Cunnane G, Youssef P, Yanni G, Fitzgerald O, Mulherin D. Microscopic measurement of synovial membrane inflammation in rheumatoid arthritis: proposals for the evaluation of tissue samples by quantitative analysis. Br J Rheumatol 1998; 37:636-42.
(4) Burmester G R, Stuhlmuller B, Keyszer G, Kinne R W. Mononuclear phagocytes and rheumatoid synovitis. Mastermind or workhorse in arthritis? Arthritis Rheum 1997; 40:5-18.
(5) Camilleri J P, Williams A S, Amos N, Douglas-Jones A G, Love W G, Williams B D. The effect of free and liposome-encapsulated clodronate on the hepatic mononuclear phagocyte system in the rat. Clin Exp Immunol 1995; 99:269-75.
(6) Carol M, Pelegri C, Castellote C, Franch A, Castell M. Immunohistochemical study of lymphoid tissues in adjuvant arthritis (AA) by image analysis; relationship with synovial lesions. Clin Exp Immunol 2000; 120:200-8.
(7) Dijkstra C D, Dopp E A, Vogels I M, Van Noorden C J. Macrophages and dendritic cells in antigen-induced arthritis. An immunohistochemical study using cryostat sections of the whole knee joint of rat. Scand J Immunol 1987; 26:513-23.
(8) Edmonds J P, Scott D L, Furst D E, Brooks P, Paulus H E. Antirheumatic drugs: a proposed new classification. Arthritis Rheum 1993; 36:336-9.
(9) Egeland T. Immunological aspects of the rheumatoid synovium. Scand J Rheumatol Suppl 1987; 66:27-33.
(10) Feldmann M, Brennan F, Maini R. Role of cytokines in rheumatoid arthritis. Ann Rev Immunol 1996; 14:397-440.
(11) Feldmann M, Maini R. Anti-TNF alpha therapy of rheumatoid arthritis: what have we learned? Ann Rev Immunol 2001; 19:163-96.
(12) Firestein G S, Zvaifler N J. Anticytokine therapy in rheumatoid arthritis. N Engl J Med 1997; 337:195-7.
(13) Fox D A. The role of T cells in the immunopathogenesis of rheumatoid arthritis: new perspectives. Arthritis Rheum 1997; 40:598-609.
(14) Hudlicky T, Rinner U, Gonzalez D, Akgun H, Schilling S, Siengalewicz P et al. Total synthesis and biological evaluation of Amaryllidaceae alkaloids: narciclasine, ent-7-deoxypancratistatin, regioisomer of 7-deoxypancratistatin, 10b-epi-deoxypancratistatin, and truncated derivatives. J Org Chem 2002; 67:8726-43.
(15) Idso S B, Kimball B A, Pettit G R III, Garner L C, Pettit G R, Backhaus R A. Effects of atmospheric CO₂ enrichment on the growth and development of Hymenocallis littoralis (Amaryllidaceae) and the concentrations of several antineoplastic and antiviral constituents of its bulbs. Am J Bot 2000; 87:769-73.
(16) Keffer J, Probert L, Cazlaris H, Georgopoulos S, Kaslaris E, Kioussis D et al. Transgenic mice expressing human tumor necrosis factor: a predictive genetic model of arthritis. EMBO J. 1991; 10:4025-31.
(17) Kinne R W, Schmidt C B, Buchner E, Hoppe R, Nurnberg E, Emmrich F. Treatment of rat arthritides with clodronate-containing liposomes. Scand J Rheumatol Suppl 1995; 101:91-7.
(18) Kinne R W, Schmidt-Weber C B, Hoppe R, Buchner E, Palombo-Kinne E, Nurnberg E et al. Long-term amelioration of rat adjuvant arthritis following systemic elimination of macrophages by clodronate-containing liposomes. Arthritis Rheum 1995; 38:1777-90.
(19) Kool J, Gerrits-Boeye M Y, Severijnen A J, Hazenberg M P. Immunohistology of joint inflammation induced in rats by cell wall fragments of Eubacterium aerofaciens. Scand J Immunol 1992; 36:497-506.
(20) Lorton D, Lubahn C, Felten S Y, Bellinger D. Norepinephrine content in primary and secondary lymphoid organs is altered in rats with adjuvant-induced arthritis. Mech Ageing Dev 1997; 94:145-63.
(21) Mikami M, Kitahara M, Kitano M, Ariki Y, Mimaki Y, Sashida Y et al. Suppressive activity of lycoricidinol (narciclasine) against cytotoxicity of neutrophil-derived calprotectin, and its suppressive effect on rat adjuvant arthritis model. Biol Pharm Bull 1999; 22:674-8.
(22) Mulherin D, Fitzgerald 0, Bresnihan B. Synovial tissue macrophage populations and articular damage in rheumatoid arthritis. Arthritis Rheum 1996; 39: 115-24.
(23) Mulherin D, Fitzgerald 0, Bresnihan B. Clinical improvement and radiological deterioration in rheumatoid arthritis: evidence that the pathogenesis of synovial inflammation and articular erosion may differ. Br J Rheumatol 1996; 35:1263-8.
(24) Ouarzane-Amara M, Franetich J F, Mazier D, Pettit G R, Meijer L, Doerig C et al. In vitro activities of two antimitotic compounds, pancratistatin and 7-deoxynarciclasine, against Encephalitozoon intestinalis, a microsporidium causing infections in humans. Antimicrob Agents Chemother 2001; 45:3409-15.
(25) Pettit G R, Melody N, Herald D L. Antineoplastic agents. 450. Synthesis of (+)-pancratistatin from (+)-narciclasine as relay. J Org Chem 2001; 66:2583-7.
(26) Pettit G R, Melody N, O'Sullivan M, Thompson M A, Herald D L, Coates B. Synthesis of 10b-(R)-hydroxy-pancratistatin via narciclasine. J Chem Soc, Chem Commun 1994; 2725-6.
(27) Pettit G R, Melody N, Simpson M, Thompson M, Herald D L, Knight J C.

Antineoplastic agents 500. Narcistatin. J Nat Prod 2003; 66:92-6.
(28) Pettit G R, Pettit G R III, Backhaus R A, Boettner F E. Antineoplastic agents, 294. Variations in the formation of pancratistatin and related isocarbostyrils in Hymenocallis littoralis. J Nat Prod 1995; 58:37-43.
(29) Pettit G R, Pettit G R III, Backhaus R A, Boyd M R, Meerow A W. Antineoplastic agents, 256. Cell growth inhibitory isocarbostyrils from Hymenocallis. J Nat Prod 1993; 56:1682-7.
(30) Pettit G R, Pettit G R III, Groszek G, Backhaus R A, Doubek D L, Barr R J et al. Antineoplastic agents, 301. An investigation of the Amaryllidaceae genus Hymenocallis. J Nat Prod 1995; 58:756-9.
(31) Pettit G R, Melody N, Herald D L, Schmidt J M, Pettit R K, Chapuis J-C. Synthesis of 10b(R)-hydroxypancratistatin, 10b(S)-hydroxy-1-epipancratistatin, 10b(S)-hydroxy-1,2-diepipancratistatin and related isocarbostyrils. Heterocycles 2002; 56:139-55.
(32) Richards P J, Williams A S, Goodfellow R M, Williams B D. Liposomal clodronate eliminates synovial macrophages, reduces inflammation and ameliorates joint destruction in antigen-induced arthritis. Rheumatol (Oxford) 1999; 38:818-25.
(33) Veronese F M, Ceriotti G, Baccichetti F, Carlassare F, Moschini F, Caliceti P et al. In vitro and in vivo behavior of narciclasine released from matrices based on poly (2-hydroxyethyl methacrylate). Farmaco 1991; 46:1061-70.
(34) Verschure P J, Van Noorden C J, Dijkstra C D. Macrophages and dendritic cells during the early stages of antigen-induced arthritis in rats: immunohistochemical analysis of cryostat sections of the whole knee joint. Scand J Immunol 1989; 29:371-81.
(35) Williams R O, Feldmann M, Maini R N. Anti-tumor necrosis factor ameliorates joint disease in murine collagen-induced arthritis. Proc Natl Acad Sci USA 1992; 89:9784-8.
(36) Yui S, Mikami M, Mimaki Y, Sashida Y, Yamazaki M. Inhibition effect of Amaryllidaceae alkaloids, lycorine and lycoricidinol on macrophage TNF-alpha production. Yakugaku Zasshi 2001; 121:167-71.

### Example 3

### Reduction of Myeloid Cells in SNS-Treated Rats

One of characteristics of inflammatory response is an elevation of myeloid cells. To determine whether SNS exert any inhibitory effect on the production or migration of these cells, we used immunostaining and flow cytometry to assess cellular distribution of splenic cells upon SNS treatment. Specifically, modulation of immune cells by SNS was demonstrated by examination of the changes in the population size of T, B, and myeloid cells using lineage-specific markers in rats receiving saline vs. SNS treatment. The cells were stained with fluorescence-conjugated antibodies specific to CD3, CD45R, and CD11b, followed by analyses using flow cytometry to reveal T, B, and myeloid cells, respectively. Lymphoid and myeloid cells were distinguished based on their differing forward and side scatter patterns.

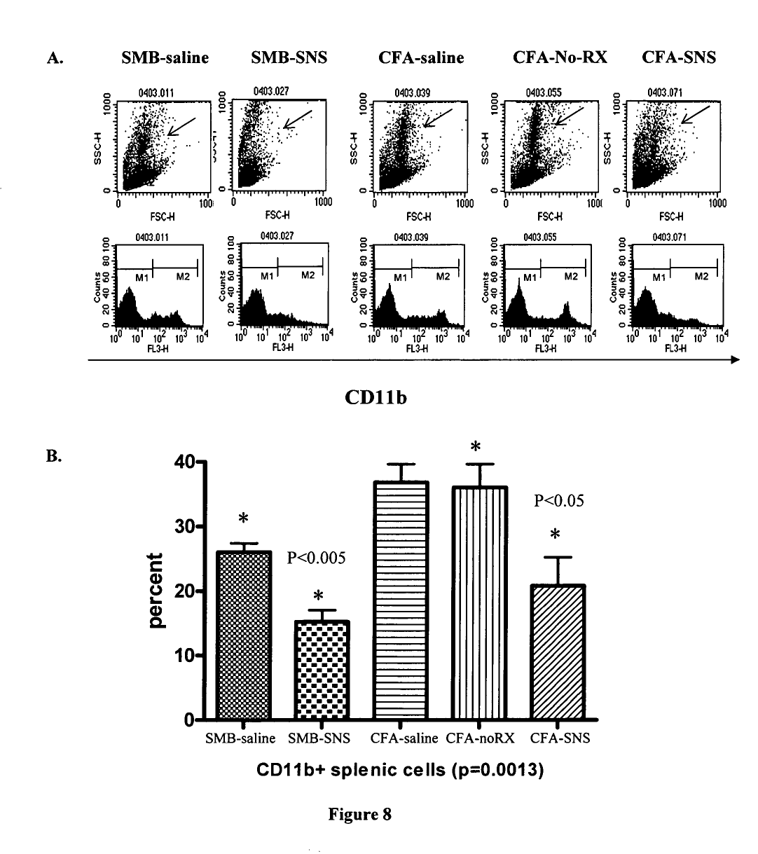

A slight increase in the number of myeloid cells in arthritic rats is commonly seen as compared to non-arthritic rats. However, SNS treatment greatly reduced this myeloid population, especially in the SNS/non-arthritic group. Further confirmation was demonstrated following analysis of the number of CD11b+ cells, in which total splenic cells were examined, including both lymphoid and myeloid populations based on the forward and size scatters. In contrast, comparable numbers of T and B lymphocytes were found in the various treatment groups. The data is summarized in FIG. 8. The data indicates that there is a reduction of CD11b positive myeloid cells in sodium narcistatin treated animals.

These preliminary findings also suggest that SNS primarily targets myeloid cells for inhibition, which may account for its ameliorating effect in the progression of an established arthritis. Thus, SNS not only demonstrates anti-inflammatory activity in vitro, it clearly suppresses the induced inflammation in vivo. This newly discovered immune-modulating activity will facilitate the application of anti-cancer drugs in inflammatory and autoimmune conditions.

### Example 4

### Collagen type II Antibody-Induced Arthritis (CAIA) Model

Collagen-induced arthritis (CIA) is an experimental model for rheumatoid arthritis. In this model, antibodies against type II collagen are critical for the pathogenesis of arthritis. Treatment with a mixture of four monoclonal anti-type II collagen mAb and lipopolysaccharide (LPS) reproducibly induced arthritis in various strains of mice. This model system has been used for investigating underlying cellular and molecular mechanisms during the effector phase of CIA (1).

We found that the pre-established CAIA mice treated with SNS showed a significant reduction in disease progression, suggesting that SNS has anti-inflammatory potential for treating Rheumatoid arthritis and other inflammatory diseases.

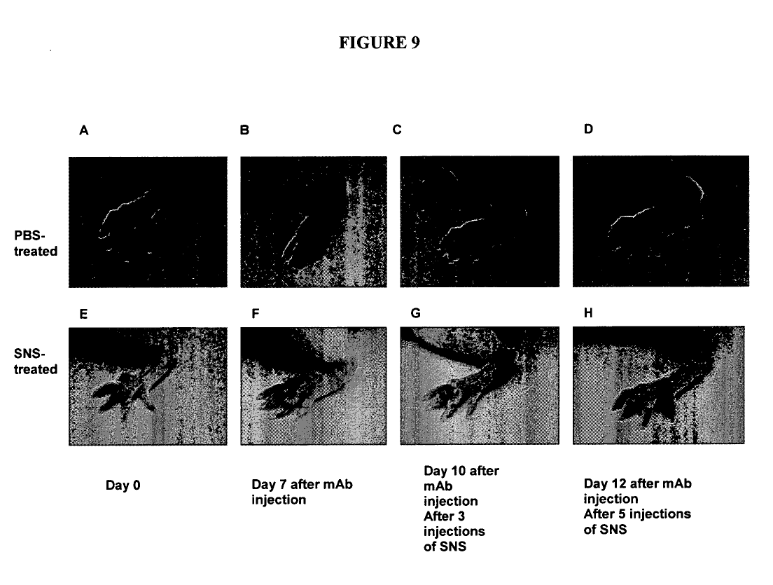

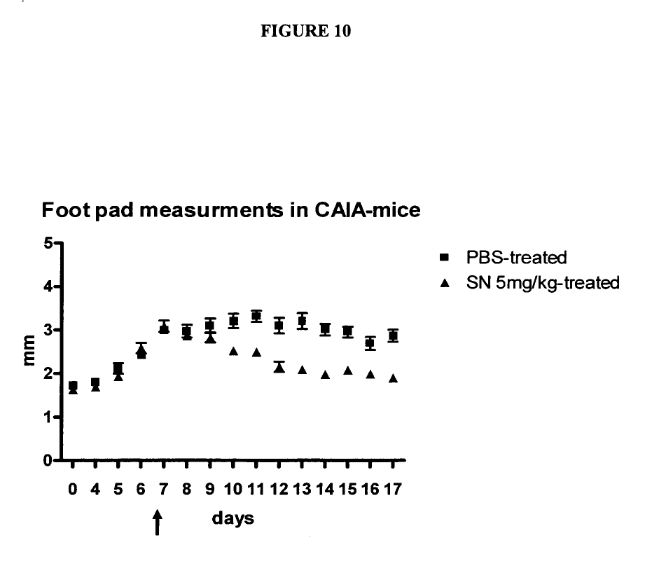

FIGS. 9-10 show CAIA progression and the effect of SNS on hind paw swelling in CAIA mice. FIG. 9a shows Disease progression in representative animals. A and E: Day 0 (mAb immunization), B and F: Day 7 after antibody injection, C. Day10 after immunization in PBS-treated CAIA, G: Day 10 after immunization in SNS-treated CAIA after three SNS injections, D: Day 12 after immunization in PBS-treated CAIA mice, and E: Day 12 after immunization in SNS-treated CAIA mice after five SNS injections. Ib. Measurements of ankle width showed a significant decrease in sodium narcistatin-treated CAIA mice compared to saline-treated CAIA mice (P<0.0001) (FIG. 10). Solid square: control CAIA group treated with PBS (n=5), closed triangle: sodium narcistatin-treated CAIA group (n=5). Values represent mean±S.E.M. p<0.0001 vs. control CAIA group.

These data show that CAIA mice treated with SNS showed reduced joint swelling after the third injection with the drug and the swelling disappeared after the fifth injection with SNS. Although SNS exhibits strong anti-inflammatory activity, it was toxic to the animals in this study at the dosage used. We did not observe toxicity if the mice only received SNS treatment. However, combination of SNS and LPS treatment appeared to increase toxicity. The observed toxicity may be caused by bone marrow depletion. We are currently devoting our effort to optimize the doses and schedules of drug delivery to minimize the toxicity while to maintain the anti-inflammatory activity of the drug.

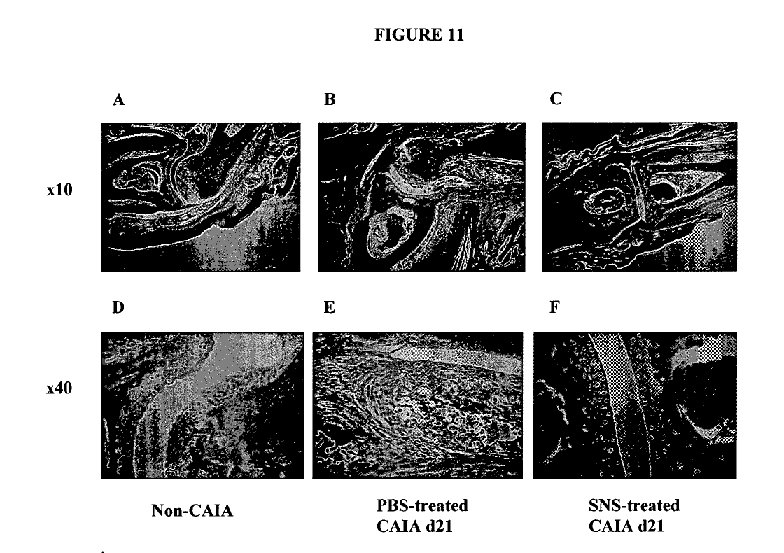

FIG. 11 shows a histological analysis of ankle joints in control, CAIA and SNS-treated CAIA mice. Sections of ankle joints were stained with hematoxylin and eosin (original magnification, ×10) on day 21 after immunization. Representative joints in normal group (A and D), control CAIA (B and E), and SNS-treated CAIA (C and F) are shown. Histological analyses of joint tissues showed very little inflammatory infiltration to ankle joints and virtually no cartilage and bone destruction in SNS-treated CAIA mice, similar to normal control mice. SNS treatment reduced infiltration of neutrophils and macrophages and destruction of cartilage and bone.

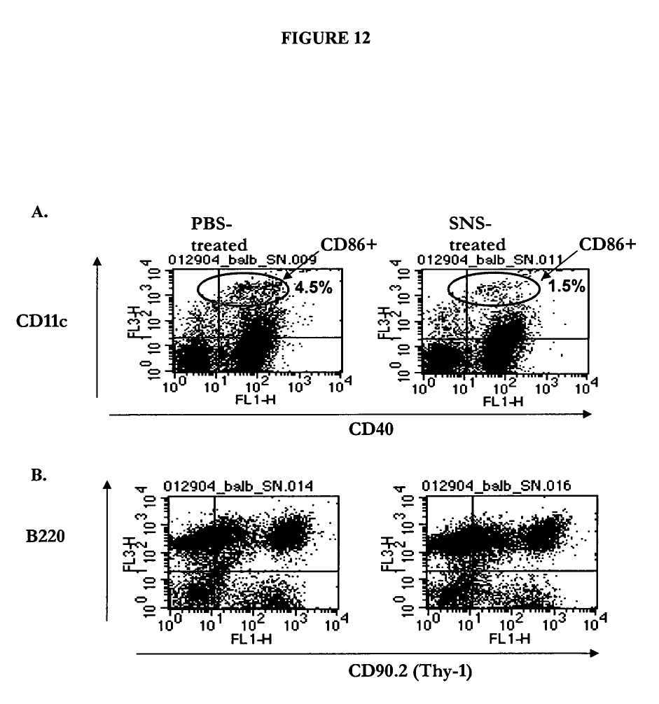

FIG. 12 shows the observed reduction of dendritic cells in Balb/c mice treated with SNS. Balb/c mice were treated i.p. with SNS for five consecutive days at 5 mg/kg and their spleen were harvested the next day after the last injection. Control mice received same numbers of injection of PBS. Splenic cells were stained with fluorescence-labeled antibodies, and analyzed by flow cytometry. A. Expression of mature dendritic cells, as revealed by surface markers, CD11c and CD40 in CD86. B. Comparable numbers of B and T cells were found between control and SNS-treated mice, based on the expression of B220 and CD90.2 (Thy-1). A reduction of CD11c+/CD86+/CD40+ spleen cells in SNS-treated mice was seen whereas T- and B-cell profile were not affected.

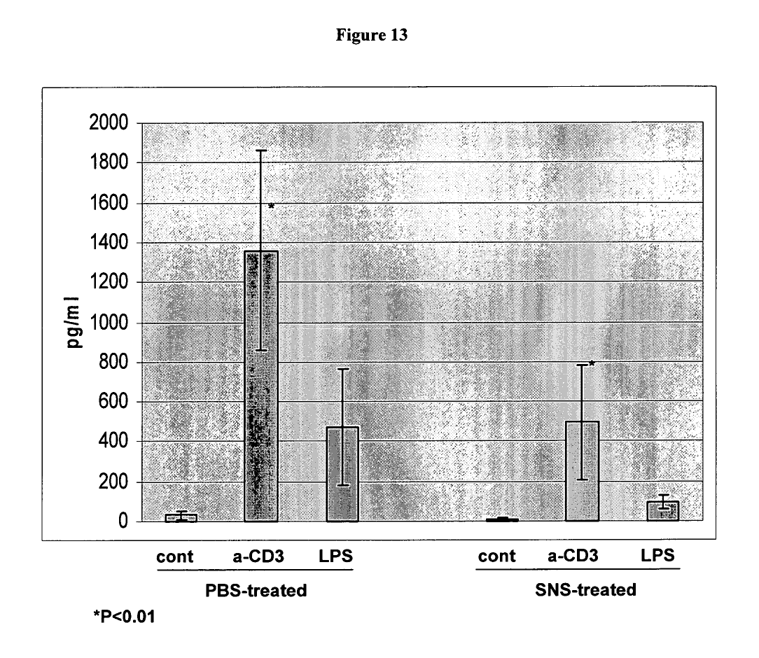

FIG. 13 demonstrates TNFα production from splenic cultures of Balb/c mice treated with SNS or PBS for 5 days. Isolated splenic cells were cultured with either immobilized anti-CD antibody (50 ug/ml) or 1 ug/ml LPS for 72 hrs. Cells without any stimulation were included as control. The supernatants from these cultures were analyzed for TNFα using BD™ CBA Mouse Inflammatory Kit (BD Biosciences), according to the manufacture's procedure. The average TNF concentration in each culture was derived from 2-3 independent experiments of 4-5 mice per group per experiment. No arthritis was induced in these experiments. A significant reduction of TNFα was found in SNS-treated splenic cells cultured with anti-CD3 (p<0.01 using t-test statistical analysis). Thus, TNFα production was reduced in the splenic culture of SNS-treated mice, in response to T cell activation by anti-CD3 antibody.

The above data show that the number of CD11c+/CD86+/CD40+ cells is reduced in spleens of SNS-treated mice. In addition, production of TNF-α in these splenocytes is decreased as compared to the splenocytes of non-SNS-treated control mice. Given the important role that dendritic cells and cytokines play in the pathological manifestation of rheumatoid arthritis, the selective inhibition of those cells and cytokines by SNS may contribute to its dramatic effect in ameliorating the disease progression in CAIA mice. Thus, SNS has therapeutic potential for treating rheumatoid arthritis and possibly other autoimmune diseases, and will be further investigated for its mode of action in suppressing inflammatory reactions.

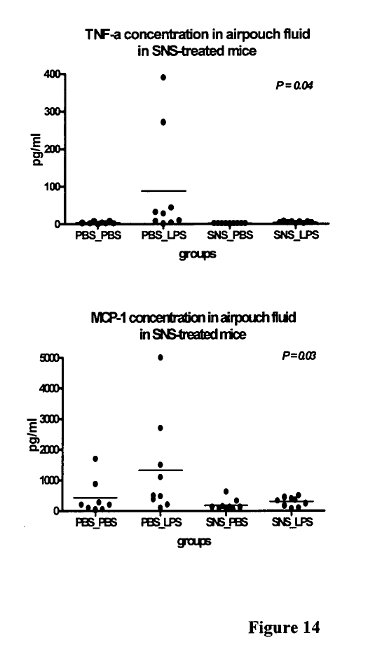

To further test effects of SNS in whole animals, we established an air-pouch assay to evaluate the effect of the compound, SNS, on LPS-stimulated cytokine production, following a procedure described previously (9). (See FIG. 14) Mice were pre-treated with PBS or SNS then challenged with LPS. The pouch wash-out was then analyzed for cytokine production using BD™ CBA Mouse Inflammatory Kit (BD Biosciences). As shown in FIG. 14, both TNFα and monocyte chemotactic protein-1 (MCP-1) were significantly reduced in SNS-treated mice as compared to the LPS-stimulated controls. This data clearly indicates that SNS treatment can suppress the production of pro-inflammatory cytokines in LPS-stimulated mice, which may account for the observed improvement in SNS-treated CAIA mice.

Summary of Example 4:

Sodium narcistatin exhibits anti-inflammatory activity in the CAIA model with reduction in infiltration of inflammatory cells, joint swelling, and joint destruction. This activity is correlated with its inhibitory effect on mature dendritic cells in spleen, and TNFα production. Thus, sodium narcistatin has therapeutic potential for treating rheumatoid arthritis and possibly other inflammatory diseases, and will be further investigated for its mode of action in suppressing inflammatory reactions.

References for Example 4

1. Nandakumar K. S., L. Svensson, and R. Holmdahl. 2003. Collagen Type II-Specific Monoclonal Antibody-Induced Arthritis in Mice. AJP. 163 (5):1827-1837.
2. Mikami M., M. Kitahara, M. Kitano, Y. Ariki, Y., Mimaki, Y. Sahida, M. Yamazaki, and S. Yui. 1999. Suppressive activity of lycoridicinol (narciclasine) against cytotoxicity of neutrophil-derived calprotectin, and its suppressive effect on rat adjuvant arthritis model. Biol Pharm Bull 22 (7):674-678.
3. Pettit, G. R., N. Melody, M. Simpson, M. Thompson, D. L. Herald, and J. C. Knight. 2003. Antineoplasitc agents 500. Narcistain. J Nat Prod. 66:92-96.
4. Lu J, Kasama T, Kobayashi K, Yoda Y, Shiozawa F, Hanyuda M, Negishi M, Ide H, Adachi M. 2000. Vascular endothelial growth factor expression and regulation of murine collagen-induced arthritis. J. Immunol. 164:5922-5927
5. Afuwape A O, Feldmann M, Paleolog E M. 2003. Adenoviral delivery of soluble VEGF receptor 1 (sFlt-1) abrogates disease activity in murine collagen-induced arthritis. Gene Ther. 10:1950-1960.
6. Lavastre, V., H. Cavalli, C. Ratthe & D. Girard. 2004. Anti-inflammatory effect of Viscum album agglutinin-1 (VAA-1): induction of apoptosis in activated neutrophils and inhibition of lipopolysaccharide-induced neutrophilic inflammation in vivo, Clin. Exp. Immunol. 137: 272-278.
7. Rojas J, Paya M, Dominguez J N, Ferrandiz M L. 2003. ttCH, a selective inhibitor of inducible nitric oxide synthase expression with antiarthritic properties. Eur J. Pharmacol. 2003 Mar. 28; 465: 183-189.
8. Rojas J, Paya M, Devesa I, Dominguez J N, Ferrandiz M L. 2003. Therapeutic administration of 3,4,5-trimethoxy-4′-fluorochalcone, a selective inhibitor of iNOS expression, attenuates the development of adjuvant-induced arthritis in rats. Naunyn Schmiedebergs Arch Pharmacol. 368:225-233.
9. Lavastre, V., H. Cavalli, C. Ratthe & D. Girard. 2004 Anti-inflammatory effect of Viscum album agglutinin-1 (VAA-1): induction of apoptosis in activated neutrophils and inhibition of lipopolysaccharide-induced neutrophilic inflammation in vivo, Clin. Exp. Immunol. 137: 272-278.

1. A method for treating an inflammatory condition comprising administering to a subject with an inflammatory condition an amount effective to treat the inflammatory condition of a compound selected from the group consisting of narcistatin, pancratistatin, pancratastatin-7′ phosphate and pancratastatin-3′,4′ cyclic phosphate, or pharmaceutically acceptable salts thereof.

2. The method of claim 1 wherein the inflammatory condition is selected from the group consisting of arthritis, inflammatory bowel disease, asthma, emphysema, ulcerative colitis, rheumatoid arthritis, juvenile chronic arthritis, Crohn's disease, Sjörgen's disease, systemic lupus erythematosus, psoriasis, sciatica, atherosclerosis, infection, strain, sprain, cartilage damage, trauma, and recent orthopedic surgery.

3. A method for treating arthritis, comprising administering to a subject with arthritis an amount effective to treat arthritis of a compound selected from the group consisting of narcistatin, pancratistatin, pancratastatin-7′ phosphate and pancratastatin-3′,4′ cyclic phosphate, or pharmaceutically acceptable salts thereof.

4. The method of claim 3 wherein the arthritis comprises rheumatoid arthritis

5. The method of claim 3 wherein the arthritis comprises osteoarthritis.

6. A method for one or more of reducing bone loss in a subject, treating a rheumatoid disease, and/or treating an autoimmune disorder, comprising administering to a subject at risk of bone loss, suffering from a rheumatoid disease, and/or suffering from an autoimmune disorder an amount effective to reduce bone loss, treat a rheumatoid disease, and/or treat an autoimmune disorder of a compound selected from the group consisting of narcistatin, pancratistatin, pancratastatin-7′ phosphate and pancratastatin-3′,4′ cyclic phosphate, or pharmaceutically acceptable salts thereof.

7. The method of claim 6 wherein the subject suffers from one or more conditions selected from the group consisting of osteoporosis, osteoarthritis, Paget's disease, humoral hypercalcemia of malignancy, hypercalcemia from tumors metastatic to bone, and periodontal disease.

8. A method for treating one or more disorders selected from the group consisting of osteoporosis, osteoarthritis, Paget's disease, humoral hypercalcemia of malignancy, hypercalcemia from tumors metastatic to bone, and periodontal disease, comprising administering to a subject with one or more of the disorders an amount effective to treat the one or more disorders of a compound selected from the group consisting of sodium narcistatin, pancratistatin, pancratastatin-7′ phosphate and pancratastatin-3′,4′ cyclic phosphate, or pharmaceutically acceptable salts thereof.

9. The method of claim 8 wherein the subject suffers from osteoporosis.

10. The method of claim 8 wherein the subject suffers from osteoarthritis.

11. The method of claim 8 wherein the subject suffers from Paget's disease.

12. The method of claim 8 wherein the subject suffers from humoral hypercalcemia of malignancy.

13. The method of claim 8 wherein the subject suffers from hypercalcemia from tumors metastatic to bone.

14. The method of claim 8 wherein the subject suffers from periodontal disease.

15. A method for reducing cellular production of one or more of vascular endothelial growth factor, nitric oxide, IL-1, MCP, and/or TNFα in a subject in need thereof comprising administering to the subject an amount effective to reduce cellular production or one or more of vascular endothelial growth factor, nitric oxide, IL-1, MCP, and/or TNFα of a compound selected from the group consisting of narcistatin, pancratistatin, pancratastatin-7′ phosphate and pancratastatin-3′,4′ cyclic phosphate, or pharmaceutically acceptable salts thereof.
